(19) United States
(12) Patent Application Publication
(10) Pub. No.: US 2007/0101461 A1
(43) Pub. Date: May 3, 2007

(54) Method for Improving Phytoremediation Treatment of a Contaminated Medium

(76) Inventors: Daniel Van Der Lelie, Geel (BE); Siegfried D'Haene, Drongen (BE); David Niall Dowling, Carlow (IE); Ulrich Karlson, Roskilde (DK); Edward R.B. Moore, Branuschweig (DE); Safiyh Taghavi, Liege (BE); Stefan A.J. Trapp, Neustadt (DE); Jaco Vangronsveld, Diepenbeek (BE)

Correspondence Address: MERCHANT & GOULD PC, P.O. BOX 2903, MINNEAPOLIS, MN, 55402-0903, US

(21) Appl. No.: 11/459,866

(22) Filed: Jul. 25, 2006

Related U.S. Application Data
(63) Continuation-in-part of application No. 11/056,502, filed on Feb. 11, 2005, now abandoned.
Application No. 11/056,502 is a continuation of application No. 10/150,884, filed on May 16, 2002, now abandoned.
(60) Provisional application No. 60/291,344, filed on May 16, 2001.

(51) Int. Cl. A01H 11/00; A62D 3/02

(52) U.S. Cl. 800/295; 435/262.5; 588/300; 588/402; 588/403

(57) ABSTRACT

The invention concerns a method for the phytoremediation treatment of a medium contaminated with at least one element selected from the group consisting of (preferably water soluble and volatile) organic pollutants, heavy metals, radionuclides or a mixture thereof, comprising the step of cultivating upon the contaminated medium a plant associated with an endophytic microorganism able to improve the phytoremediation of the plant, to reduce phytotoxicity of chemicals. The invention further relates to methods for improving phytoremediation by directly modifying members of the endogenous endophytic community of a plant, via horizontal gene transfer. Another part of the invention relates to plants associated with such endophytes and/or plants with a modified endogenous endophytic community.

### CROSS-REFERENCE

This application is a continuation-in-part of U.S. application Ser. No. 11/056,502, which was a continuation of U.S. application Ser. No. claims benefit from U.S. application Ser. No. 10/150,884, filed May 16, 2002.

### FIELD OF THE INVENTION

The present invention is in the field of biotechnology and is related to the use of endophytic microorganisms, especially bacteria to improve phytoremediation of a contaminated medium, especially soils contaminated by heavy metals, radionuclides and/or organic pollutants.

### BACKGROUND

The soil pollution by toxic organic compounds is an important environmental problem. Phytoremediation may offer a possible solution or reduction of the problem. Phytoremediation is the process of using plants for in situ remediation of soils or groundwater contaminated with different pollutants via extraction, degradation and/or stabilization of contaminants. Phytoremediation of organic xenobiotics is based on combined action between plants and their associated microorganisms. Degradation of organic contaminants can occur in the plant rhizosphere and in planta.

The use of biological techniques can strongly reduce the costs of remediating sites contaminated with organic xenobiotics. For large contaminated sites, bioremediation is the only alternative economically and socially acceptable. Especially phytoremediation, one of the soft bioremediation techniques, is becoming an acceptable alternative for the treatment of contaminated sites and wastewater. Phytoremediation of organic contaminants is based on the combined action between plants and their associated microorganisms, such as mycorrhizal fungi and bacteria.

Degradation of organic xenobiotics can occur in the plant rhizosphere and in planta. However, certain organic pollutants may not be degraded, but may be accumulated in the plant or be volatilised through the plant leaves.

In addition, water soluble and volatile organic pollutants might be partially degraded by plants and subsequently, accumulation of toxic metabolites can occur.

Soil contaminants, especially organic xenobiotics with a log Kow between 0.5 and 3.5 and weak electrolytes (weak acids and bases or amphoteres such as herbicides) are readily taken up by plants (Trapp et al., 1994; Trapp 2000). Recent unpublished evidence suggests that numerous compounds (see also Table 1) enter the xylem faster than the soil microflora can degrade them, even if the rhizosphere is enriched with degrader bacteria.

TABLE 1
Non-exhaustive list of pollutants that provide potential
problems in phytoremediation due to uptake followed
by insufficient plant metabolism.

| | Fate in plant | |
|---|---|---|
| | (toxic, build up, | |
| Compound | or volatile) | Reference |
| Phenols | Toxic | Pfleeger et al., 1991 |
| Chloro-phenols | Toxic | Pfleeger et al., 1991 |
| TNT | Toxic, degraded to | Thompson et al., 1998 |
| | amino-dinitrotoluene | |
| Amino-dinitrotoluene | Rather persistent, | Thompson et al., 1998 |
| | toxic | |
| MTBE | Volatile | Trapp et al., 1994 |
| BTEX | Volatile | Trapp et al., 1994 |
| TCE | Volatile, Build-up | Trapp et al., 1994 |
| | of trichloroacetate | |
| PER | Volatile | Trapp et al., 1994 |

Although some pollutants are metabolized by plants, numerous pollutants—or their metabolites—are toxic to plants. This can seriously limit the applicability of phytoremediation (because plants do not grow correctly or may die in toxic soils). Alternatively, in the case of volatile pollutants, plants release these compounds, or their metabolites, through the stomata, which questions the merits of an efficient phytoremediation by the plant.

Although offering some interesting benefits compared to the traditional remediation techniques, phytoremediation of contaminated medium by phytoextraction of heavy metals and radionuclides still has its limitations. A suitable plant used in extraction of heavy metals should possess several characteristics, which are rarely found within one plant species. For this reason, different strategies are currently being investigated in order to improve crops for phytoextraction processes (Cunningham & Berti, 1993; Cunningham & Ow, 1996; Burd et al., 1998; Arazi et al., 1999; Brewer et al., 1999).

Endophytic microorganisms, especially bacteria are ubiquitous in most plant species, residing latently or actively colonising plant tissues. Historically, endophytic microorganisms, especially bacteria have been thought to be weakly virulent plant pathogens, but have recently been discovered to have several beneficial effects on host plants, such as plant growth promotion and increased resistance against plant pathogens and parasites.

Endophytic bacteria have been isolated from different parts of the plants, including roots, stems and leaves. Endophytic colonisation of the vascular system (phloem, xylem) has been reported, their numbers being quite significant (10+3-10+6 cfu/ml). Especially in trees, such as poplar or willow that are currently used to develop phytoremediation strategies for organics, the time period between uptake of organics by the roots and their arrival in the leaves takes several hours to days (Mc Crady et al., 1987; Trapp et al., 2001), as the compounds travel through the vascular system.

### SUMMARY OF THE INVENTION

The present invention is related to a method for the phytoremediation treatment of a medium (such as a soil or an aqueous medium), contaminated with at least one element selected from the group consisting of (preferably, water soluble or volatile) organic pollutants, heavy metals or radionuclides, the method comprising the step of cultivating in or upon the medium, a plant associated with an endophytic microorganism able to improve phytoremediation by the plant and the step of recovering the element or the degraded metabolites of the element inside the plant.

According to the invention the endophytic microorganism present in the plant is an endophytic bacteria (or an endophytic fungi).

Endophytic microorganisms, especially endophytic bacteria are defined as those microorganisms that are able to enter plant tissues and to establish themselves inter-and intra-cellularly (Di Fiori & Del Gallo, 1995). They have the ability to establish an active relationship with their hosts and can be defined as colonists (Kloepper & Beauchamp, 1992; Kloepper et al., 1992).

The endophytic microorganism could be a isolated and purified natural microorganism or a genetically modified microorganism.

In one embodiment, the endophytic microorganisms are present in the vascular system (phloem, xylem) or the root system of the plant.

According to a preferred embodiment of the present invention, the endophytic microorganism is selected but not limited to the group consisting of the genera Pseudomonas, Azotobacter, Azomonas, Acinetobacter, Xanthomonas, Stenotrophomonas, Comamonas, Burkholderia, Ralstonia, Alcaligenes, Derxia, Xylella, Delftia, Rhizobium, Bradyrhizobium, Rhizomonas, Sphingomonas, Azospirillum, Blastomonas, Porphyrobacter, Zymomonas, Brevundimonas, Phenylbacterium, Agrobacterium, Chelatobacter, Sinorhizobium, Allorhizobium, Phyllobacterium, Aminobacter, Mesorhizobium, Ochlrobactrum, Beijerinckia, Azorhizobium, Devosia, Nevskia, Afipia, Blastobacter, Chromobacterium, Herbaspirillum, Acidovorax, Brachymonas, Variovorax, Thauera, Zoogloea, Azoarcus, Spirillum, Rhodanobacter, Halomonas, Alcanivorax, Zymobacter, Agromonas, Chryseomonas, Flavimonas, Phenylbacterium, Rhizobacter, Moraxella, Psychrobacter, Alteromonas, Pseudoalteromonas, Shewanella, Vibrio, Photobacterium, Aeromonas, Enterobacter, Pantoea, Brenneria, Pectobacterium, Bacillus, Actinomyces, Corynebacterium, Frankia, Nocardia, Rhodococcus, Streptomyces, Flavobacterium, Flexibacter, or the group of the pink-pigmented facultatively methylotrophic bacteria, including Methylobacterium populi.

According to a preferred embodiment of the present invention the water soluble or volatile organic pollutant is an agrochemical such as a pesticide or an herbicide such as 2,4-D, or a pollutant such as naphthalene, toluene, benzene, phenols, chlorophenols, nitro aromatic compounds such as 2,4-DNT, TNT, amino-dinitrotoluene, MTBE, BTEX, chlorinated ethenes, organotin compounds, PCBs, PBBs, brominated flame retardants, fluorinated alkylsulfonates, in particular perfluoro-octanyl sulfonate (PFOS).

According to another embodiment of the present invention, the heavy metals or radionuclides are metals selected from the group consisting of zinc, cadmium, cobalt, nickel, copper, lead, mercury, thallium, barium, boric, selenium, chrome, cesium, strontium, uranium, plutonium, lanthanides or their salts.

Another aspect of the present invention is related to a plant comprising in its vascular system (phloem, xylem) a genetically modified, or a naturally occurring isolated and purified, endophytic microorganism that is able to express proteins that allow an efficient degradation or a phytoaccumulation of at least one of the elements above-mentioned (organic pollutants, heavy metals, radionuclides, or a mixture thereof).

However, by introducing endophytic organisms that express degradation genes for specific organic xenobiotics, these compounds might be efficiently degraded, resulting in no or strongly reduced build-up of these compounds or their toxic degradation intermediates in the plants or in reduced phytovolatilization.

Endophytic micro-organisms equipped with the appropriate degradation pathway help plants survive under conditions of elevated levels of the organic pollutant. If the endophyte is able to metabolize (degrade or treat) the (organic) pollutant in question, the plant automatically benefits therefrom.

If it is known that the organic pollutant is degraded into a toxic degradation product, it is advisable to select natural strains that are able to degrade the start and the intermediate (toxic) compound and/or to construct the endophytic micro-organism with degradation pathways for both compounds.

Introduction and heterologous expression of known heavy metal resistance genes in endophytic microorganisms, especially endophytic bacteria resulting unexpectedly in an effect on the uptake capacities of heavy metals by their host plant. Salt et al. (1999) have shown that Cd tolerant rhizobacteria are able to promote Cd precipitation processes near the root surface of Indian mustards plants and consequently decreased the toxic effects of the metal cation for the roots. Previous studies have shown that several mechanisms can be responsible for bacterial heavy metal resistance e.g. blocking the entry of toxic ions in the cells, intracellular sequestration of the metals by metal binding proteins, enzymatic conversion of the metal to a less toxic form and energy driven efflux systems for cations and anions encoded by resistance genes, such as the czc, cnr, nec, cad, and ars operons (Mergeay, 1997; Taghavi et al., 1997).

Bio-precipitation and sequestration processes also seem to take place when bacteria are equipped with efflux mechanisms. This phenomenon was observed in cultures of Ralstonia metallidurans CH34 (previous Alcaligenes eutrophus CH34) when grown in the presence of high concentrations of Cd or Zn and attributed to the action of the czc resistance operon on the pMOL30 plasmid (Diels et al., 1995). Such bio-preceipitation and sequestration characteristics could offer interesting benefits for the bacteria, and in the case of endophytic bacteria the speciation of the heavy metals might be altered in the host plant from a free to a less available form and lead to a reduced toxicity of the heavy metals on plant metabolism.

Advantageously, a selection pressure is present or applied. Such selection pressure means a selective advantage for the endophytic inoculum, which can often be seen as a starter culture that allows horizontal gene transfer of the desired properties or allows other bacteria that are better adapted to the local conditions to gradually replace the original population of (engineered) endophytic micro-organisms.

Advantageously, the degradation or treatment pathway can be passed on from the endophyte of the inoculum to the natural endophytic community of the plant via horizontal gene transfer. The endogenous endophytic micro-organism may receive the desired (metabolic) properties (via horizontal gene transfer) from an endophytic organism with which the plant is inoculated. In an embodiment, the desired metabolic properties are located on a broad host range conjugative plasmid that is able to replicate in members of the endogenous endophytic community.

A further aspect of the invention relates to a method of directly adapting the metabolic capabilities of a plant's endogenous endophytic population without the need of first selecting the appropriate endophytic micro-organisms from the plant species of interest.

Further provided is a method for improving phytoremediation by (directly) adapting the metabolic capabilities of a plant's endogenous endophytic population, the method comprising the steps of
Equipping a broad-host-range plasmid with the genetic information encoding the desired metabolic properties, and
Bringing the plant into contact with a donor strain containing the plasmid to ensure transfer of the desired properties within the endogenous endophytic community of the plant. This transfer is possible via horizontal gene transfer.
In an embodiment, the donor strain is an endophytic micro-organism able to enter the plant, however, without the need of establishing itself among the endogenous endophytic community. In a further embodiment, the donor strain is a bacterium. In various embodiments, selection pressure is present or applied as selective advantage for the (starter) endophytic population with the desired characteristics.

The invention further relates to a plant comprising in its endogenous endophytic community at least one endophytic species that has received the desired metabolic properties via horizontal gene transfer. Preferably the modified (endogenous) endophtyic micro-organism is present in the vascular system and/or the roots of the plant. Such plant will have improved phytoremediation capacities compared to a similar non-modified plant.

Yet a further aspect of the invention concerns a method for the phytoremediation treatment of a medium contaminated with at least one element selected from the group consisting of organic pollutants, heavy metals, radionuclides or a mixture thereof, comprising the step of cultivating upon the contaminated medium a plant comprising in its endogenous endophytic community at least one endophytic species that has received (via horizontal gene transfer) the genetic information encoding for the degradation, treatment, phytoextraction and/or the accumulation of this element. Preferably this element is an organic pollutant and the genetic information received encodes for the appropriate degradation pathways.

The present invention will be described in more details in the following non-limiting examples in reference to the enclosed FIG. 1.

### DESCRIPTION OF THE DRAWINGS

FIG. 1 represents an Ni concentration (mg/kg dry weight) in roots and shoots of Lupinus luteus L. plants for different inocula and following 0.25mM NiCl₂ treatment (L.S.2.4 was inoculated as the wild type strain and L.S.2.4::ncc-nre as its Ni resistant derivative. Data are mean values of 3 replicate samples ±S.D. Different letters indicate values that are statistically significant (P<0.05)).

FIG. 2 shows the total amount of toluene (μg) detected in Tenax traps connected with the upper compartment (containing the aerial part of Lupinus luteus plant) determined by GC-MS. For this experiment, non inoculated control plants and lupine plants inoculated with B. cepacia strains VM1330, BU0072 and G4 were used. The statistical significance of the results was confirmed at the 5% level using a a-way ANOVA model.

FIG. 3 shows the difference in biomass (g) between inoculated and controls yellow lupine plants, before and after adding toluene. Plants were grown as hydroponics in a glass cuvette system. M0: plant weight (g) before addition of toluene; Mt: plant weight (g) 96 hours after toluene addition. The concentrations of toluene were 0, 100, 500, and 1000 mg/l, respectively. Standard deviations are indicated as bars. The statistical significance of the results was confirmed at the 5% level using a two-way ANOVA model, separately exploring treatment (bacterial inoculums) and toluene doses.

FIG. 4 shows the biomass (g) of Yellow lupine plants, grown in non-sterile sandy soil under the greenhouse conditions, after 14 days exposure to the different toluene concentrations. Plants were irrigated every other day with half-strength Hoagland's solution to which toluene was added at concentrations of 0, 100, 250, and 500 mg/liter. Standard deviations are indicated as bars. The statistical significance of the results was confirmed at the 5% level using a two-way ANOVA model, separately exploring treatment (bacterial inoculums) and toluene doses.

FIG. 5 shows the phytotoxic effect of toluene on Yellow lupine grown in non-sterile sandy soil under the greenhouse conditions. The labels indicate the control plants and lupine plants inoculated with B. cepacia strains VM1330, BU0072 and G4. Plants were irrigated every other day with half-strength Hoagland's solution to which toluene was added at concentrations of 0, 100, 250, and 500 mg/liter (indicated as ppm). A picture of representative plants was taken after 14 days of irrigation with toluene containing solutions

FIG. 6 shows the influence of different concentration of toluene on growth of Lupinus luteus.

FIG. 7 shows roots of Pea plants after exposure to 54 mg 2,4-D. Little or no enlargement or thickening was observed on the inoculated plants (A). Note the enlargement of root tips in the control plants (B).

FIG. 8 shows amounts of 2,4-D remaining in the stem/leaf tissue of exposed plants (A) and in the soil (B).

FIG. 9: (A) P. putida VM1450 biofilm within the rhizosphere of inoculated plants exposed to 54 mg 2,4-D. (B) P. putida VM1450 cells in the rhizosphere of inoculated plants exposed to 27 mg 2,4-D. (C) P. putida VM1450 micro-colony within the root of inoculated plants exposed to 54 mg 2,4-D. (D) Micro-colony of P. putida VM1450 within the stem of plants exposed to 27 mg 2,4-D.

FIG. 10 shows transpiration rates of uninoculated plants (A) compared to inoculated plants (B) when naphthalene is applied.

FIG. 11 shows healthy Pea plants showing phytoprotection against naphthalene when inoculated with an endophytic micro-organism capable of degrading naphthalene (B). The top row shows non-inoculated plants (A).

FIG. 12 shows Pea endophytes to which the naphtalene degradation pathway present on the plasmid pNAH7 could be transferred via horizontal gene transfer.

FIG. 13 shows plant growth parameters for poplar cuttings calculated after 10 weeks of growth in the presence or absence of toluene (Tol) (0 or 500 mg/l). (A) Growth indexes calculated as (Mt−M0)/M0, where M0 is the plant weight (in grams) before addition of toluene and Mt is the plant weight (in grams). (B) Root and leaf weight. The data are the means of 10 replicates; standard deviations are indicated by error bars. The statistical significance of the results was confirmed at the 5% level using a two-way analysis of variance model, separately exploring treatment (bacterial inocula) and toluene doses.

FIG. 14 shows the total amount of toluene (μg) released from poplar into the upper cuvette compartment. The amount of evapotranspired toluene was calculated per square centimeter of leaf area. The data are the means of three replicates; standard deviations are indicated by error bars.

### DESCRIPTION

The present invention aims to provide a new method and plant for improving phytoremediation, especially for water soluble and volatile organic pollutants degradation by plant and to improve treatment of toxic pollutants or their metabolites by the plant without being toxic for the plant. Another aim of the invention is to reduce the possible volatilization through the plant's leaves of the pollutants and their possible metabolites.

The methods of the present invention include selecting an endophytic microorganism containing degradation genes for one or more organic pollutants; and cultivating a plant associated with the endophytic microorganism upon a medium contaminated by one or more organic pollutants, thereby improving the phytoremediation of the organic pollutant from the contaminated medium by the plant.

As described above various pollutants have been found susceptible to degradation by microorganisms. Various examples include agrochemicals including herbicides, such as 2,4-Dichlorophenoxyacetic acid; nitraromatic compounds, such as 2,4-DNT, TNT, and amido-dinitrotoluene; volatile organic compounds such as MTBE, BTEX, TCE or PER; polyaromatic hydrocarbons such as phenol, chlorophenol, toluene, benzene, and naphthalene; and other organic compounds, such as chlorinated ethenes, organotin compounds, PCBs, PBBs, brominated flame retardants, and fluorinated alkylsulfonates, including perfluoro-octanyl sulfonate (PFOS).

The methods of the present invention are compatible with plants serving as host to an endophytic community. For example, most broad leaf plants have endophytic communities of microorganisms associated with either or both their root and vascular structure. Examples of broad leaf plants includes but is not limited to poplar Populus, willow (Salix), and pea (Pisum salivium). Other examples of plants compatible with the inventive methods are described herein.

### EXAMPLES

### Example 1

### Heavy Metal Sequestration by Natural and Genetically Modified Endophytic Bacteria

Pseudomonas sp. VM422 was isolated as an endophytic strain from surface sterilised Brassica napus. This strain was selected for its zinc resistance phenotype: VM422 had a MIC value for zinc of 20 mM on Tris minimal medium (Mergeay et al., 1985). This strain was tested for its zinc complexing capacity by growing it for 66 hours in liquid medium in the presence of 60000 μg/l ZnCl₂. After the incubation period, approximately 800 μg/l Zn remained in the solution: the majority of the Zn was biosequestrated around the VM422 cells. For comparison, a similar experiment with Ralstonia metallidurans CH34, a well-known heavy metal sequestration bacterium (Diels et al., 1995), resulted in a decrease of Zn to 2730 μg/l in the remaining solution. This experiment demonstrates the feasibility to use natural, heavy metal resistant endophytic bacteria for heavy metal sequestration from solution.

Burkholderia cepacia L.S.2.4::ncc-nre, in which the miniTn5-ncc-nre transposon had been introduced (Taghavi et al, 2001) was examined for its efficiency for nickel sequestration. Strain L.S.2.4 was grown for 7 days in liquid medium in the presence of 25 mg/l NiCl2. After the incubation period, approximately 15 mg/l Ni remained in the solution, indicating that 40% of the Ni was sequestrated around the B. cepacia L.S.2.4.:ncc-nre cells. This experiment demonstrates the feasibility to use genetically modified, heavy metal resistant endophytic bacteria for heavy metal sequestration from solution.

### Example 2

### Construction of Recombinant Endophytic Strains Equipped with Degradation Pathways for Specific Organic Xenobiotics

For construction of strains of endophytic bacteria with improved degradation capacity of organic xenobiotics (benzene, toluene, phenols and TCE) natural gene transfer was used. Natural gene transfer is based on bi or tri parental conjugation or exogenous plasmid isolation.

As a model endophytic strain to be equipped with degradation pathways was used a nickel-kanamycin marked derivative of Burkolderia cepacia L.S.2.4 named strain BU 0072, which was constructed at VITO (Taghavi, S. et all,2001). Burkolderia cepacia L.S.2.4 has yellow lupine (Lupinus luteus L.) as host.

As a donor strain for degradation pathway Burkolderia cepacia G4 (TOM, conjugative plasmid, tol+) was used.

Donor strain and receptor strain were grown overnight in LB medium, washed in 10-2 MgSO4 and aliquots of 100 μl were added to a sterile filter (0.45 μl) and incubated overnight at 30° C. on solid LB medium. The filter containing donor, receptor and transconjugants were washed in a 10-2 MgSO4 solution in order to release the bacteria. Transconjugant was selected by means of their acquired Ni+Km+Tol resistance on minimal Tris buffered medium supplemented with the appropriate concentration of selective marker i.e. 1 mM NiCl2, 100 μg/ml Km and toluene as carbon source.

After three weeks the mating between BU 0072 and BU G4 resulted in transconjugants that were KmR and tol+, with a transfer efficiency of 7.1×10-3.

The presence of nre which confers the Ni resistance in the transconjugants of BU 0072×G4 was confirmed with PCR using the following specific primers:

Sense:

[SEQ ID NO:1]

5′-ggAgAgCgCCgTgACCCAggCgAAgAAggCgCTggTgCATgACCATA
TCgACC-3′
Antisense:

[SEQ ID NO:2]

5-CACgATCACCATggCgCTggCTgCTgCCgCggCAAGATTCAgCgCCAg
CAgTCCC TTC-3′

The strain BU0072 was used as a positive control.

Amplification was carried out as follows: a preliminary denaturation step was done at 95° C. for 10 min, followed by 35 cycles of 2 min at 95° C., 1 min at 55° C., 2 min at 72° C. and 8 min at 72° C. PCR product of 930 bp was checked by electrophoresis in 1.2% agarose gels. All the transconjugants showed the nre specific fragment.

The presence of pTOM was confirmed by plasmid isolation of the transconjunants and of Burkolderia cepacia G4 as positive control, while strain BU0072 was used as a negative control. A large fragment corresponding with the plasmid was present in the transconjugants and in G4, but was lacking in BU0072. One of the representative transconjugants was chosen and named VM1330.

Strain VM1330 is used to reinoculate lupine plants to prove the concept of the project.

### Example 3

### Development and Comparison Techniques for Efficient Reinoculation of Endophytic Strains in Their Host Plants

After having marked and equipped endophytic bacteria with degradation pathways an efficient recolonization of host plant is an important prerequisite to evaluate their contribution inside of the plant to degrade the pollutants as they are being transported trough the plant and consequently reduce phytotoxicity and volatilization of the pollutants.

Preparation of Bacterial Inoculum:

A VM 1330 strain was grown in 284 tris buffered, salted, minimal liquid medium with addition of 0.2% gluconate at 22° C. on rotary shaker for a period of 7 days. Next, inoculum was centrifuged at 6000 rpm during 15 minutes, washed twice in MgSO4 −2. Inoculum was diluted and plated on 284 medium with addition of 1 mM Ni, 50 mg/l kanamicyne and toluene in order to test the purity of the solution and the presence of Ni, Km and toluene resistance characteristics.

Seeds Surface Sterilization:

Seeds of Lupinus luteus were surface sterilized in solution containing 1% active chloride and 1 droplet Tween per 100 ml solution. After sterilization seeds were rinsed 3 times in sterile water and dried on sterile filter paper. In order to test sterilization efficiency seeds were incubated on 869 medium during 3 days at 30° C.

Inoculation and Plant Growth Conditions:

Surface sterile seeds of Lupinus luteus were planted in sterile plastic jars (800 ml), completely filled with sterile perlite and saturated with ½ concentrated sterile Hoagland's solution. Five seeds were planted in each jar.

Perlite was chosen as plant growth substrate because it can be sterilized easily and provides the roots with moisture, nutrients and a good aeration due to the large surface area and the physical shape of each particle.

The bacterial inoculum was added in each jar at concentration of 108 colony forming units (CFU) per milliliter Hoagland's solution. Inoculum was added in MgSO4 −2, whereas for the non-inoculated plants the same amount of MgSO4 −2 was added.

The jars were covered with sterile tinfoil in order to allow a good bacterial colonization and prevent contamination and dispersion of the inoculated bacteria through the air. After germination of the seeds, holes were made in the tinfoil and plans were grown through the holes.

Plants were grown for 21 days in climate chamber with constant temperature 22° C., relative humidity 65% and 12 hour light and dark cycle.

Recovery of Bacteria:

After 21 days plants were harvested. Roots and shoots were separated. Fresh root and shoot material was vigorously washed in distilled water, sterilized in solution containing 1% active chloride supplemented with 1 droplet Tween per 100 ml solution, rinsed 3 times in sterile distilled water. After sterilization roots and shoots were macerated using mixer in 10 ml sterile MgSO4 −2. 100 μl of supernatant was immediately plated on three different media: 284+0.2% gluconate; 284+1 mM Ni+100 Km+0.2% gluconat and 284+1 mM Ni+50 Km+toluene.

From the perlite growth substrate 2.5 g was shaken for 30 minutes in 10 ml MgSO4 −2 and plated on the same media as mentioned before. Incubation for 7 days at 30° C. proceeded the bacterial counting.

Results of the counting are presented in table 2.1

Those bacteria were purified on the same media and it was shown that bacteria from reinoculated plants could grow on media with addition of 1 mM Ni, Kanamicyne and toluene. None bacteria from control plant have that capability.

Presence of Ni and Km resistance and presence of Tom plasmide in bacteria isolated from reinoculated plants of Lupinus luteus is confirmed by means of BOX PCR (Vito).

TABLE 2.1
Number of bacterial colonies isolated from roots and shoot of reinoculated
and control plants of Lupinus luteus. Numbers in parentheses are the
numbers of different colony forming units, eye spotted.

| | Bacteria | Bacteria | Bacteria | Bacteria |
|---|---|---|---|---|
| | isolated | isolated | isolated | isolated |
| | from the | from the | from the | from the |
| | roots of | shoots of | roots of | shoots of |
| | reinoculated | reinoculated | control | control |
| | plants | plants | plants | plants |
| 284 + gluc | 314(3) | 438(2) | 193(4) | 71(4) |
| 284 + 1 mM Ni + | 0 | 11(1) | 0 | 0 |
| 100 Km + gluc | | | | |
| 284 + 1 mM Ni + | 0 | 0 | 0 | 0 |
| 50 Km + tol | | | | |

### Example 4

### Improved Phytoaccumulation of Heavy Metals

In order to exploit the use of endophytic bacteria to improve the phytoextraction of heavy metals, Burkholderia cepacia was selected as endophytic strain. Some B. cepacia strains have been reported as facultative endophytes of lupine plants or were found to colonise roots of various maize cultivars (Hebbar et al., 1992a; Hebbar et al., 1992b).

Wild type strain Burkholderia cepacia L.S.2.4 and its nickel resistant derivative L.S.2.4::ncc-nre (Taghavi et al., 2001) were inoculated in perlite and the sterile Lupinus seedlings were grown on this substrate for 21 days under controlled environmental conditions. Non-inoculated sterile plants were used as controls. In the absence of NiCl₂, no difference in growth response was observed between the 21 days-old non-inoculated control plants and the inoculated lupine plants when the shoot biomass and length were considered. The roots seemed to be slightly but significantly affected in their growth when B. cepacia was added as a wild type strain or as the nickel resistant derivative, indicating that the presence of B. cepacia L.S.2.4 has a minor, but positive effect on the root development in the absence of nickel. The nickel concentration in both roots and shoots was measured with A.A.S but was below the detection limit (<2.5 mg/kg DW).

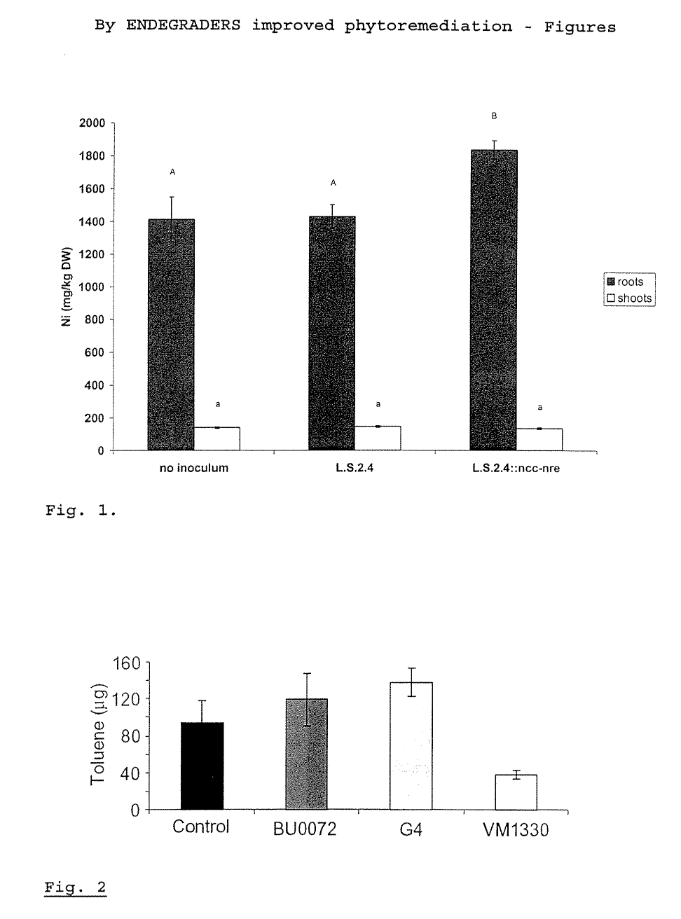

Addition of 0.25 mM NiCl₂ to the perlite resulted in a decrease of the growth parameters when compared to the treatment without NiCl₂, suggesting a toxic effect of the nickel cations. The presence of B. cepacia, both wild type and its nickel resistant derivative, did not influence the growth of the plants. No significant differences were observed when root and shoot biomass and length were measured. However, a different response in nickel accumulation was observed when the nickel concentration in the roots was compared for the different treatments (FIG. 1). A significantly higher total nickel concentration was measured in the lupine roots inoculated with the nickel resistant B. cepacia L.S.2.4::ncc-nre, while the non-inoculated control plants and the plants inoculated with the wild type strain L.S.2.4 had similar but lower nickel contents. In contrast to the roots, the nickel concentration in the shoots was comparable for the different treatments (FIG. 1). This is explained by the preferential colonization by B. cepacia L.S.2.4 of the Lupinus roots. Another reason might be that the nickel ions are complexed by B. cepacia L.S.2.4 in the roots, and consequently their transfer to the shoots is blocked.

### Example 5

### Decreased Phyto-Volatilisation of Toluene and TCE

Many degradation pathways for organic xenobiotics are located on mobile genetic elements, including plasmids and transposons. Therefore it should be feasible to use natural gene transfer, either based on bi- or tri-parental conjugation or exogenous plasmid isolation (Szpirer et al., 1999), for constructing strains of endophytic bacteria with improved degradation capacity of organic xenobiotics. An example is the transfer the TOM_{31c} plasmid, enabling growth on phenol and toluene, from B. cepacia G4 (Shields et al., 1995) into the Ni-resistance strain B. cepacia L.S.2.4::ncc-nre. Transconjugants should be selected on their ability to grow on toluene in the presence of 2 mM Ni. In addition the transconjugants will be able to degrade TCE (tri-chloro-ethylene) without induction of the tomA gene by toluene, due to the constitutive expression of the toluene-ortho-monooxygenase (Shields and Reagin, 1992).

In order to reduce phyto-volatilization of organic xenobiotics through the plants' stoma, it is suggested to inoculate plants with endophytic bacteria. These bacteria should preferentially colonize the xylem, and should be able to degrade the organic contaminant of interest, such as those mentioned in Table 1. Either natural endophytic bacteria, able to degrade the organic contaminant of interest, or endophytic bacteria equipped either by natural gene transfer or recombinant DNA techniques, can be selected for this purpose.

In case the internal plant concentration of the organic xenobiotic will be too low to efficiently induce the degradation pathway or when the contaminant is degraded due to co-metabolism, mutated pathways that show constitutive expression of degradation should be preferentially introduced in the endophytic bacteria. An example is constitutively expressed toluene-ortho-monooxygenase of B. cepacia G4 (Shields and Reagin, 1992), which results in constitutive TCE degradation without the need to induce TCE cometabolism by the presence of phenol or toluene.

For phytoremediation of contaminated groundwater, preferentially deep-rooting trees are used, such as poplar or willow. In addition to their large water consumption, trees have the advantage that the retention time of the contaminant in the xylem is quite long (up to two days) allowing sufficient time for in planta degradation of the contaminant by the endophytic bacteria.

Improved phytoremediation of toluene and TCE, resulting in decreased phyto-volatilization, would involve the following steps:
Selection of the plant species of interest, well adapted to the local climate and geohydrological constraints;
Isolation of endophytic bacteria from the selected plant species;
a Selection or construction of toluene degrading strains or construction of recombinant stains that constitutively express the degradation pathway, including a toluene-ortho-monooxygenase that allows efficient degradation of TCE without the need of cometabolism.
Inoculation of the selected plants with the endophytic bacteria that are now equipped with the necessary degradation pathways.
The overall outcome will be improved phytoremediation of toluene and TCE due to increased in planta degradation and reduced phyto-volatilisation.
A similar concept is feasible for any organic xenobiotic that is taken up by plants and for which microbial degradation pathways are available and can be expressed in plant associated endophytic bacteria.
   Employment of Reinoculated and Not Reinoculated Plants in Experiments with Toluene

Reinoculated and not reinoculated plants of Lupinus luteus were grown for a period of three weeks in sterile plastic jars, filed with sterile perlite saturated with ½ concentrated sterile Hoagland's solution. Plants were grown in climate chamber, with constant temperature 22° C.; relative humidity 65% and light and dark cycles of 12/12 (described earlier).

After three weeks plants were carefully taken out from jars, all perlit was removed and plant roots were vigorously rinsed in sterile water. Rinsing of the roots in sterile water was done in order to prevent degradation of toluene by bacteria outside the plant. From one reinoculated plant and from one control plant roots and shoots were separated, sterilized for 5 minutes in solution containing 1% active chloride, rinsed three times in sterile water and mixed in 10 ml MgSO4 −2. 100 μl of the supernatant was plated on three different media (284+gluc; 284+1 mM Ni+100 Km+gluc; 284+1 mM Ni+50 Km+toluene).

Subsequently, two reinoculated and one non-reinoculated plant were settled in three separated glass grow chambers. The growchambers have dimensions 29 cm height and diameter 9 cm. Compartment above and compartment under are separated with a glass plate, which have insertion breadth as tree of Lupinus luteus. In each chamber one plant was placed, insertions were closed with gyps so that shoots were in the upper compartment and roots in the lower compartment. Those two compartments were completely separated with no gas exchange between them. The lower compartment was filed with 300 milliliters of sterile, ½ concentrated Hoagland's solution. Each compartment was connected with air source with inflow of 1 liter per hour. Furthermore, each compartment was fitted with a two-linked Tenax traps. Traps were used to capture any transpired or volatilized toluene and were changed at 1 hour to 24 hours intervals. Toluene was added on the beginning of the experiment in the Hoagland's solution in certain concentration.

TABLE 1
Overview of representative endophytic strains found in association
with poplar plants and able to degrade toluene

| | | Toluene | tomA4 |
|---|---|---|---|
| Strain ID | Species | phenotype | PCR |
| BU61 | B. cepacia | Tol⁺ | + |
| VM1468 | B. cepacia | Tol⁺ | + |
| W604 | Enterobacter sp. | Tol⁺ | + |
| W607 | Stenotrophomonas maltophilia | Tol⁺ | + |
| W619 | Pseudomonas putida | Tol⁺ | + |
| W630 | Pseudomonas putida | Tol⁺ | + |
| W633 | Ochrobactrum sp. | Tol⁺ | + |
| W635 | Stenotrophomonas maltophilia | Tol⁺ | + |
| S645 | Pseudomonas putida | Tol⁺ | + |
| S649 | Stenotrophomonas maltophilia | Tol⁺ | + |
| S656 | Stenotrophomonas maltophilia | Tol⁺ | + |
| S662 | Enterobacter sp. | Tol⁺ | + |
| S671 | Enterobacter sp. | Tol⁺ | + |
| S672 | Pseudomonas putida | Tol⁺ | + |
| T675 | B. cepacia | Tol⁺ | + |
| R571 | Pseudomonas putida | Tol⁺ | + |
| R572 | Stenotrophomonas maltophilia | Tol⁺ | + |
| R591 | Stenotrophomonas maltophilia | Tol⁺ | + |
| R599 | Pseudomonas putida | Tol⁺ | + |

Experiment Number 1

Toluene was added in concentration of 1000 mg/liter.

The experiment was running for 5 days. Columns with Tenax were changed every 24 hours.

Biomass of the plants was measured at the beginning and on the end of the experiment. Results are presented in table 2.2:

TABLE 2.2
Biomass of the reinoculated and control plants before
and after experiment where toluene was added in concentration
1000 mg/l. Growth index = (Mt − M0)/Mt

| Growth | | biomass | biomass | | | Growth |
|---|---|---|---|---|---|---|
| chamber | Plant | day 1 | day 5 | Difference | | index |
| 1 | Reinoculated | 7.26 g | 9.26 g | 2 | g | 0.28 |
| 2 | Reinoculated | 7.15 g | 9.17 g | 2.02 | g | 0.28 |
| 3 | Control | 7.62 g | 8.42 g | 0.8 | g | 0.10 |

Biomass of reinoculated plants increased in five days for 2 grams, while biomass of control plant increased 0.8 grams.

In order to determine success of the reinoculation, the bacteria were isolated from reinoculated and not reinoculated plants not used in the experiment. Once the experiment was finished, the bacteria were also isolated from the plants used in the experiment. The results are presented in table 2.3 and 2.4. Only in the inoculated plants bacteria able to grow on toluene as sole carbon source could be isolated.

TABLE 2.3
Number of bacterial colonies isolated from roots and shoot
of reinoculated and control plants of Lupinus luteus not
used in experiment. Numbers in parentheses are the numbers
of different colony forming units, eye spotted.

| | |
|---|---|
| Reinoculated plant | Control plant |

| Media | Root | Shoot | Root | Shoot |
|---|---|---|---|---|
| 284 + gluc | 952(4) | 74(7) | ∞(3) | 62(2) |
| 284 + 1 mM Ni + | 0 | 11(1) | 0 | 0 |
| 100 Km + gluc | | | | |
| 284 + 1 mM Ni + | 0 | 58(1) | 0 | 0 |
| 50 Km + toluene | | | | |

TABLE 2.4
Number of bacterial colonies isolated from roots and shoot
of reinoculated and control plants of Lupinus luteus used
in experiment. Numbers in parentheses are the numbers of
different colony forming units, eye spotted.

| | |
|---|---|
| Reinoculated plant | Control plant |

| Media | Root | Shoot | Root | Shoot |
|---|---|---|---|---|
| 284 + gluc | ∞(3) | 401(4) | ∞(1) | 1330(3) |
| 284 + 1 mM Ni + | 8(1) | ∞(1) | 0 | 0 |
| 100 Km + gluc | | | | |
| 284 + 1 mM Ni + | 13(1) | ∞(1) | 0 | 0 |
| 50 Km + toluene | | | | |

Concentration in Tenax traps was measured by means of GC-MS. Results are presented in table 2.5.

TABLE 2.5
Amount of toluene in μg detected
in Tenax traps, means by GC-MS

| | | Compartment above | | Compartment under | |
|---|---|---|---|---|---|
| | Measurement | First | Control | First | Control |
| | in hours | trap | trap | trap | trap |
| Reinoculated | 24 | 11.4 | | 154.5 | |
| plant 1 | 48 | 9.3 | | 126 | |
| | 72 | 0.9 | | 89.4 | |
| | 96 | 78.1 | | 82.6 | |
| | 120 | 5.2 | 14.6 | 104.5 | 110.7 |
| | | 104.9 | 14.6 | 557 | 110.7 |
| | | 119.5 | | 667.7 | |
| | | 787.2 | | | |
| Reinoculated | 24 | 37.2 | | 269.6 | |
| plant 2 | 48 | 1.3 | | 121.7 | |
| | 72 | 1.3 | | 97 | |
| | 96 | 15.5 | | 68 | |
| | 120 | 8.5 | 4.5 | 82.5 | 129.8 |
| | | 63.8 | 4.5 | 638.8 | 129.8 |
| | | 68.3 | | 768.6 | |
| | | 836.9 | | | |
| Control plant | 24 | 0.2 | | 1.4 | |
| | 48 | 0.3 | | 68.1 | |
| | 72 | 42.9 | | 75.1 | |
| | 96 | | | | |
| | 120 | 0.2 | 5.4 | 10.4 | 24.7 |
| | | 43.6 | 5.4 | 155 | 24.7 |

| | |
|---|---|
| 49 | 179.7 |

228.7

Experiment Number 2

Toluene was added in concentration of 500 mg/liter.

The experiment was running 3 days. Columns with Tenax were changed every 24 hours.

Biomass of the plants was measured at the beginning and at the end of the experiment. Results are presented in table 2.6:

TABLE 2.6
Biomass of the reinoculated and control plants before and after
experiment where toluene was added in concentration 500 mg/l

| Growth | | Biomass | Biomass | | Growth |
|---|---|---|---|---|---|
| chamber | Plant | day 1 | day 3 | Difference | index |
| 1 | Reinoculated | 6.99 g | 8.6 g | 1.66 | 0.23 |
| 2 | Reinoculated | 7.15 g | 9.01 g | 1.86 | 0.26 |
| 3 | Control | 7.44 g | 8.56 g | 0.92 | 0.15 |

Biomass of reinoculated plants increased in three days by nearly 2 grams, while biomass of control plant increased 1.12 grams.

Bacteria were isolated from reinoculated and not reinoculated plants not used in experiment as well as from the plants used in experiment after experiment was finished. Results are presented in table 2.7 and 2.8.

TABLE 2.7
Number of bacterial colonies isolated from roots and shoot
of reinoculated and control plants of Lupinus luteus not
used in experiment. Numbers in parentheses are the numbers
of different colony forming units, eye spotted.

| | |
|---|---|
| Reinoculated plant | Control plant |

| Media | Root | Shoot | Root | Shoot |
|---|---|---|---|---|
| 284 + gluc | ∞(4) | 314(4) | ∞(5-6) | 39(5) |
| 284 + 1 mM Ni + | 29(1) | 16(1) | 3(1) | 0 |
| 100 Km + gluc | | | | |
| 284 + 1 mM Ni + | 79(1) | 23(1) | 124(1) | 2(1) |
| 50 Km + toluene | | | | |

TABLE 2.8
Number of bacterial colonies isolated from roots and shoot
of reinoculated and control plants of Lupinus luteus used
in experiment. Numbers in parentheses are the numbers of
different colony forming units, eye spotted.

| | Reinoculated plant | | Control plant | |
|---|---|---|---|---|
| Media | Root | Shoot | Root | Shoot |
| 284 + gluc | ∞(2) | ∞(2) | ∞(?) | ∞(4-5) |
| 284 + 1 mM Ni + | 116(3) | 1055(2) | 0 | 0 |
| 100 Km + gluc | | | | |
| 284 + 1 mM Ni + | ∞(1) | ∞(1) | 899(1) | 77(1) |
| 50 Km + toluene | | | | |

Toluene concentration in Tenax traps was measured by means of GC-MS. Results are presented in table 2.9.

TABLE 2.9
Amount of toluene in μg detected
in Tenax traps, means by GC-MS

| | | Compartment above | | Compartment under | |
|---|---|---|---|---|---|
| | Measurement | First | Control | First | Control |
| | in hours | trap | trap | trap | trap |
| Reinoculated | 24 | 27.3 | 91.6 | 106.2 | 307.7 |
| plant 1 | 48 | 22.1 | 6.7 | 128.7 | 134.5 |
| | 72 | 11.9 | 1.6 | 121.2 | 141.1 |
| | | 61.3 | 99.9 | 356.1 | 583.3 |
| | | 161.2 | | 939.4 | |
| | | 1100.6 | | | |
| Reinoculated | 24 | 31.6 | 115.4 | 138.6 | 346.0 |
| plant 2 | 48 | 13.1 | 3.2 | 154.3 | 219 |
| | 72 | 3.4 | 0.2 | 129.8 | 173.5 |
| | | 48.1 | 118.8 | 422.7 | 738.5 |
| | | 166.9 | | 1161.2 | |
| | | 1328.1 | | | |
| Control plant | 24 | 4.2 | 0.7 | 142.9 | 285.2 |
| | 48 | 1.7 | 0.1 | 125.8 | 195.9 |
| | 72 | 0.3 | 0 | 98.1 | 144 |
| | | 6.2 | 0.8 | 366.8 | 625.1 |

| | |
|---|---|
| 7 | 991 |

998

Experiment Number 3

Toluene was now added in a concentration of 100 mg/liter.

The experiment was running 4 days. Columns with Tenax were changed regularly. To optimize the Tenax adsorption capabilities, the traps were cooled with dry ice. All experiments were done in triplicate to allow statistical analysis of the data using ANOVA.

In this experiment, the behavior of B. cepacia BU0072 (Taghavi et al, 2001; Lodewijckx et al, 2001), which is derived from the endophytic strain B. cepacia L.S.2.4., and its toluene-degrading derivative VM1330 were compared with that of the toluene-degrading soil bacterium B. cepacia G4 (a toluene-degrading rhizobacterium).

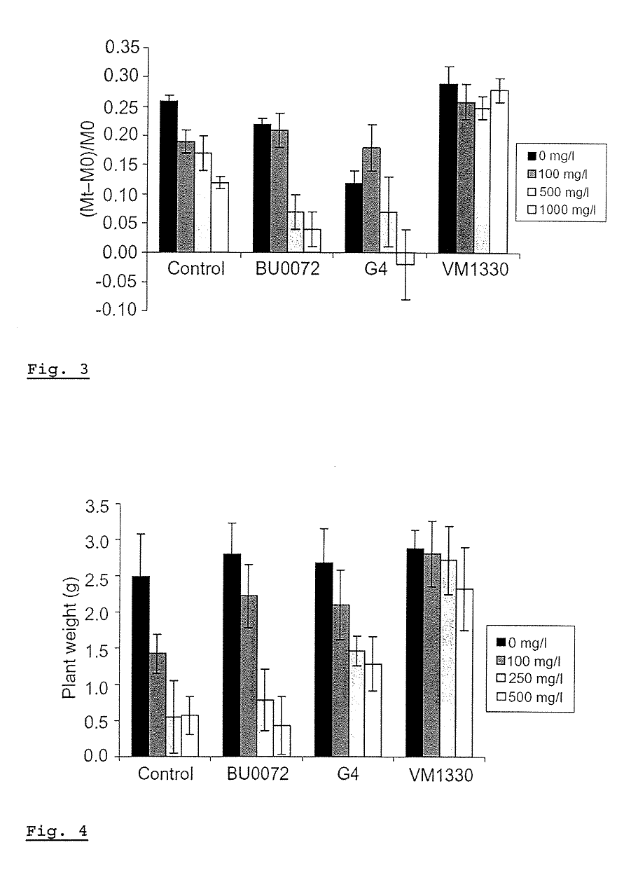

After adding toluene at a sub-phytotoxic concentration of 100 mg/l, the amount of toluene that is evapotranspired through the aerial parts of the plant (upper compartment) as well as its disappearance from Hoagland's nutrient solution (lower compartment) were measured using GC-MS. FIG. 2 shows the total amount of toluene (μg) detected in the upper compartment.

Toluene Degradation and Evapotranspiration

Compared to control plants and plants inoculated with B. cepacia BU0072 or G4, those inoculated with B. cepacia VM1330 released 50-70% less toluene in the upper compartment.

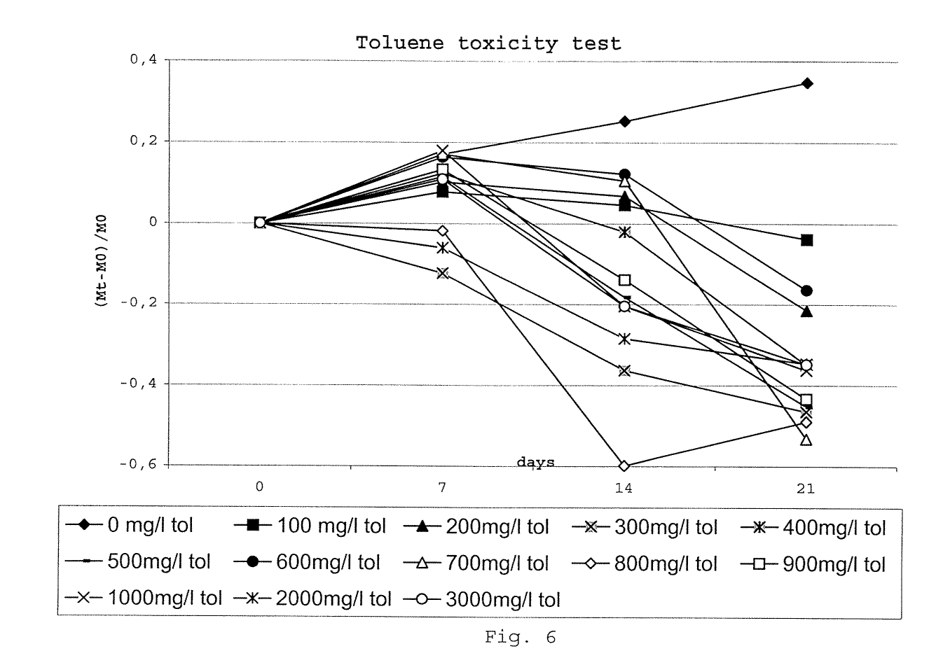

This result shows that this toluene-degrading endophytic strain not only protects its host plant against phytotoxicity (FIG. 5), but also significantly (P=0.05) lowers toluene evapotranspiration through the aerial parts, even at levels that are not toxic to control plants.

No significant differences in the concentrations of evapotranspired toluene were observed between plants inoculated with BU0072 or G4, and the non-inoculated controls. The statistical significance of the reduced toluene release in the presence of strain VM1330 was confirmed at the 5% level using a one-way ANOVA model.

Together the endophytic strain VM1330 and its host plant, yellow lupine, improve the degradation of toluene, lowering both its phytotoxicity and release by evapotranspiration.

A concentration of 100 mg/l is well above toluene concentrations as found in contaminated sites (up to 10,000 μg/l max.) A reduced phytotoxicity due to degradation of toluene in planta reflects in a higher biomass production by the plants inoculated with the endophyte compared to control plants and compared to plants inoculated with a rhizobacterium (see below and see also Tables 2.2 and 2.6).

Effect on Plant Growth During Hydroponic Cultivation and During Greenhouse Studies

For the tests with plants during hydroponic cultivation, experiments were carried out in a glass cuvette system as described above. For the greenhouse studies, plants were grown on a non-sterile sandy soil.

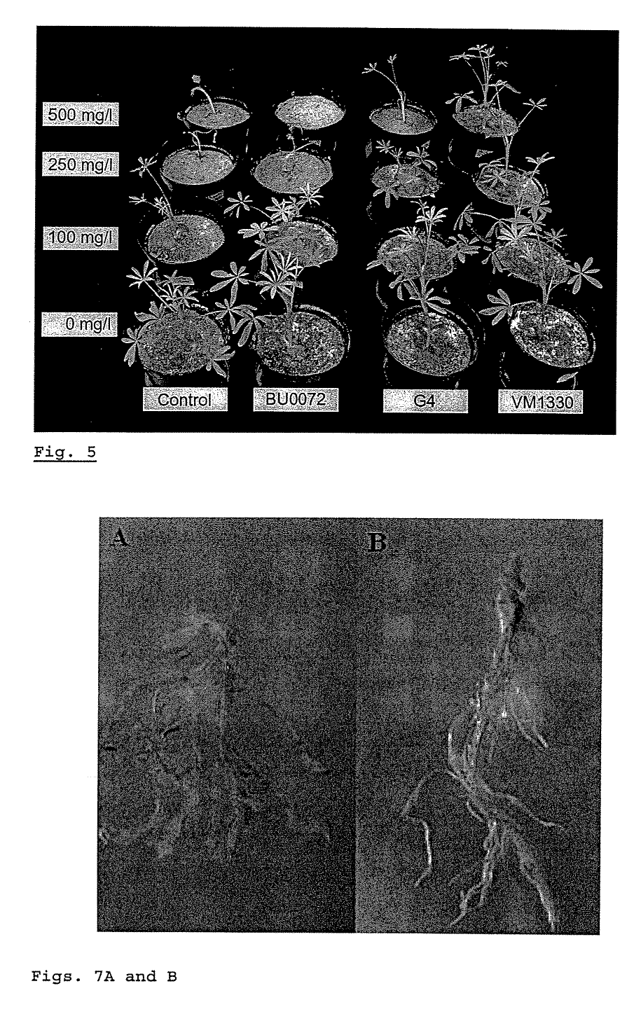

Results are shown in FIGS. 3 and 4. In both graphs, growth indices were calculated as the difference in plant's fresh weight between the onset of the experiment and the end of the experiment (96 hours and 14 days, respectively).

For plants and bacteria incubated in the presence of toluene, the growth indices suggest that increasing levels of toluene result in greater phytotoxicity.

Plants inoculated with VM1330 grew in hydroponic doses of toluene up to 1,000 mg/l without reduction in vigor, whereas non-inoculated controls showed growth reduction with as little as 100 mg/l toluene. Plants inoculated with either BU0072 and G4 had reductions in growth by 500 mg/l (FIG. 3). In greenhouse studies, plants inoculated with VM1330 and dosed with 500 mg/l for 2 weeks were still vigorous, whereas non-inoculated plants and plants inoculated with BU0072 were dead or dying. The root colonizing G4 imparted some increased tolerance to toluene but plant mass was ≈50% less than the VM1330 inoculated plants (FIG. 4). The statistical significance of the results was confirmed at the 5% level using a two-way ANOVA model, separately exploring treatment (bacterial inocula) and toluene doses.

The above data show that plants grown in the presence of toluene have 2 important characteristics imparted by the new endophyte. First, the toxicity of toluene to the plants decreased significantly. The second major change in the plants is the reduction in volume of toluene transpired.

A major concern of phytoremediation systems working on volatile solvent contamination has been the fact that the plants can transpire these compounds through the leaf stomata or stem lenticels. The fact that plants inoculated with VM1330 survive on toluene levels of 500 mg/l toluene and higher allows plant growth on sites with pollution levels above the normal phytotoxicity threshold. The methods of the invention are thus highly advantageous.

Endophtyic bacteria, when equipped with the appropriate degradation pathway, can help plants survive under conditions of elevated levels of toluene and improve phytoremediation capacities of a plant. In planta degradation of toxic compounds has a clear positive effect on plant health, vigor, growth and functionality. It is further clear that endophytic bacteria improve these properties to a level well beyond that of rhizobacteria, which can not enter the plant and consequently can not metabolize any toluene taken up by the plant.

It is clearly shown here that the combination of natural endophytic behavior plus the presence of a degradation capacity is highly advantageous.

Toxicity Test: Influence of Different Concentrates of Toluene on Growth of Lupinus luteus

During three weeks, 21 day old Lupinus luteus plants were grown hydroponicly in the presence of a different concentration of toluene. The system was open and therefore nutrient solution was changed the toluene was added every day. The concentration of toluene added to nutrient solution was 0, 100, 200, 300, 400, 500, 600, 700, 800, 900, 1000, 2000 and 3000 milligrams per liter. The biomass of the plant was measured at the day 0, 7, 14 and 21. Results are presented in the graphic 1.

FIG. 6 shows the influence of different concentration of toluene on growth of Lupinus luteus. Mt biomass of the plant on the day 7, 14 or 21. M0 biomass of the plant on day 0.

This experiment has shown that the exposure of the plant to the lowest, as well as to the highest concentration of the toluene has little influence on the grow of the plants during the first week of experiment. During the second week of the experiment, plants exposed to the highest concentration of toluene had significant retention of growth, what is expressed in the lost of biomass. At the end of the third week, all plants were death except the control plant and plant exposed to toluene at concentration of 100 mg per liter nutrient solution.

### Example 6

### Decreased Accumulation of Tri-Chloro-Acetate (TCA) as Metabolite of TCE Degradation

Certain organic xenobiotics are partially degraded by plants. However, their efficient remediation is hindered by the accumulation of toxic degradation product. An example is the degradation of TCE by several wild plants (like oak, castor bean and others), which results in the accumulation of phytotoxic TCA (tri-chloro-acetate). The in planta accumulation of TCA and consequently its phytotoxicity might be reduced by inoculation with endophytic bacteria able to degrade TCA. Improved phytoremediation of TCE due to reduced accumulation of phytotoxic TCA would involve the following steps:
Selection of the plant species of interest, well adapted to the local climate and geohydrological constraints;
Isolation of endophytic bacteria from the selected plant species;
Selection or construction of TCA degrading strains or construction of recombinant stains that constitutively express the TCA degradation pathway.
Inoculation of the selected plants with the endophytic bacteria that are now equipped with the necessary TCA degradation pathway.
The overall outcome will be improved phytoremediation of TCE due to reduced accumulation of phytotoxic TCA and hence survival of trees on higher-contaminated soils or groundwater.
A similar concept is feasible for any organic xenobiotic that is taken up by plants and for which partial degradation products, microbial degradation pathways are available and can be expressed in plant associated endophytic bacteria.

### Example 7

### Decreased Toxicity of Organic Xenobiotics, Including Agrochemicals such as Herbicides

Many herbicides, e.g. of the triazin type or 2,4-dichlorophenoxyacetic acid (2,4-D), are xylem mobile, which means they are taken up by roots and are transported through the xylem to the leaves, where they enfold their action (e.g., inhibition of photosynthesis). 2,4-D functions as a systemic herbicide that can be used to control a range of broad leaf weeds. It is normally applied as a foliar spray and although its exact mode of action is unknown it is transported through the xylem and phloem and through the roots.

Plant-specific endophytic degrader bacteria might be introduced to agricultural crop plants, which are not resistant to the herbicidal action. Endophytical degradation of compounds might therefore be used to make plants selectively resistant against broad-spectrum herbicides.

Bacterial pathways are available for the metabolism of specific organic herbicides e.g. 2,4-dichlorophenoxyacetic acid (2,4-D). Naturally occurring plasmids have been identified that encode genes for the biodegradation and detoxification of 2,4-D (Don and Pemberton, 1981).

Pseudomonas sp. strain ADP initiates catabolism of atrazine (a triazin type herbicide) via three enzymatic steps, encoded by atzA, -B, and -C, which yield cyanuric acid, a valuable nitrogen source for many bacteria and plants. Plasmid transfer studies indicated that the atzA, -B, and -C genes are localised on a 96-kb broad host-range, self-transmissible plasmid, pADP-1, in Pseudomonas sp. strain ADP (de Souza et al., 1998).

The construction and use of endophytic bacteria that can biodegrade herbicides and their inoculation on plants may confer on the plant host the ability to resist and detoxify the herbicide. This may provide many benefits including an alternative approach to generating herbicide resistant plants, allow the use of specific herbicides to promote the establishment of phytoremediation plants for in situ bioremediation, and allow the application of existing or future herbicides to a broader range of agricultural plants.

Improved phytoremdiation by the use of herbicide degrading endophytic bacteria would involve the following steps:
Isolation of endophytic bacteria from the selected plant species.
Selection or construction of specific herbicide degrading endophytic strains by natural plasmid transfer, e.g. transfer of pJMP5 from Ralstonia eutropha JMP365 (Don and Pemberton, 1985) to obtain 2,4-D degradation, or pADP-1 from Pseudomonas sp. strain ADP (de Souza et al., 1998) to obtain atrazine degradation.
Inoculation of the selected plants with the endophytic bacteria that are now equipped with the necessary degradation pathway, e. g. for 2,4-D or atrazine.
The overall outcome will be improved phytoremediation systems with herbicide degradation/resistance properties, e.g. for 2,4-D or atrazine. Another expected outcome is the selectivity of inoculated agricultural plants to broad-spectrum herbicides, e.g. glyphosate, bromacil and others.

### Example 8

### Bacterial Endophyte Enhanced Phytoremediation of the Organo-Chlorine Herbicide 2,4-Dichlorophenoxy Acetic Acid

The organo-chlorine compound 2,4-dichlorophenoxy acetic acid (2,4-D) is used as a selective systemic herbicide for the control of annual and perennial broad leaf weeds. It is one of the most commonly used herbicides in the world. The mobility of 2,4-D in soil frequently results in the contamination of not only the soil where they were applied but also to non-target surface and ground waters.

Once applied to the soil 2,4-D is usually degraded within a few days, through a combination of abiotic and biological mechanisms. However, there are reports of high levels of 2,4-D residues (0.62 mg/kg or 2% of the applied 2,4-D) remaining in treated soil for extended periods of time and observations of phytotoxic effects on crops grown in soil nine months after 2,4-D application.

Materials and methods

Materials

2,4-D (98%) was purchased from Aldrich (UK). Strain VM1450 is a chromosomally labeled (mini-Tn5 insertion of gfp:Km^{R}) derivative of P. putida strain POPHV6, a natural endophytic isolate from the stem sap of poplar trees [Germaine et al, 2004].

Pea seeds (Pisum sativum var Early Onward, Suttons Seeds Torbay, UK) were selected as model plant because of their fast growth and xenobiotic sensitive nature. Pea, like willow and poplar, are broad leaf plants.

Plant Inoculation and Application of 2,4-D

P. putida VM1450 was grown in LB broth containing 100 μg/ml Km, at 30° C., 200 rpm, to an approximate absorbance (A₆₀₀) value of 1.0. Cells from 3 ml of culture were harvested by centrifiguation, washed twice in 0.85% sterile saline and re-suspended in a final volume of 3 ml saline. This inoculum contained between 10⁵-10⁶ cells/ml as determined by standard plate count.

20 μl of the inocula was pipetted onto the surface of re-hydrated Pea seeds. The seeds were allowed to dry for 30 min before planting. Five pots containing 150 g of sterile soil were set up in duplicate. The soil was collected from the campus grounds at Carlow, classified by a sandy loam soil (60% sand, 33% clay and 7% silt) and was autoclaved twice for 2 hours at 121° C. before use in experiments.

Five inoculated seeds were then planted in each pot. The plants were allowed to develop for 4 weeks at 20-25° C. under a 16 h light/8 h dark regime and watered twice a week with 60 ml of modified Plant Nutrient Solution (PNS) ISO 8692.

In week 5, duplicate pots received 60 ml PNS containing 0, 1, 2, 4 or 8 mg/l 2,4-D. In week 6, the respective pots received 60 ml PNS containing 0, 10, 20, 40 or 80 mg/l 2,4-D. Finally, in week 7 the pots received 60 ml PNS containing 0, 100, 200, 400 and 800 mg/l 2,4-D. [0133]These 2,4-D concentrations responded to an approximate total of 0, 7, 13, 27 and 54 mg 2,4-D (this was equivalent to 0, 47, 87, 180 and 360 mg/kg soil) applied to respective pots over the course of the three weeks. Non-inoculated seeds received identical treatment and acted as controls.

Four days after the initial application of 2,4-D the plants were harvested and analyzed for 2,4-D accumulation and signs of phyto-toxic effects of 2,4-D exposure.

Determination of Biomass and Chlorophyll Levels

Four days after plants received the final 2,4-D dose they were removed from their pots and the soil was removed from the roots. The cumulative weight of the five plants from each pot was determined by weighing. Chlorophyll analysis was performed by the method of Huang et al (2004).

Briefly, 1 g samples of the leaves were removed from each plant and the chlorophyll was extracted by grinding each sample in a pestle and mortar containing 100 ml of 80% acetone solution. The mixture was then filtered, transferred to a 100 ml volumetric flask and made up to the mark with 80% acetone solution. The solution was mixed well before reading the absorbance at both 645 nm and 663 nm.

Chlorophyll levels were calculated (based on fresh weight) using the following equations (Huang et al, 2004):
Chlorophyll a(mg/g tissue)=(12.7×Abs₆₆₃ₙₘ)−(2.69×Abs₆₄₅ₙₘ)
Chlorophyll b(mg/g tissue)=(22.9×Abs₆₄₅ₙₘ)−(4.68×Abs₆₆₃ₙₘ)
Analysis of leaves from each treatment was carried out in triplicate.
2,4-D Extraction and HPLC Analysis

2,4-D extraction from soil: 2 ml HPLC grade methanol was added to 1 g soil samples and vortexed vigorously for 5 min. The samples were then centrifuged for 5 min at 14,000 rpm, the supernatant was carefully removed and centrifuged once more. The supernatant was then filtered through a 0.22 μm filter prior to HPLC analysis. Spiked samples of soil were kept refrigerated fro 7 days prior to analysis, to determine the recovery rate. Analysis of soils from each pot was carried out in triplicate.

2,4-D extraction from plant material: 5 g of plant stem material was ground in 20 ml 20 mM phosphate salt buffer (PBS) (0.55 g/l NaH₂PO₄.H₂O, 2.85 g/l Na₂PO₄, 9.0 g/l NaCl, pH 4.8) using a pestle and mortar.

The samples were then vortexed vigorously for 5 min before centrifuging at 13,000 rpm for 15 min. The supernatant was carefully removed and passed through a C₁₈ Sep-Pak™ cartridge (Waters, UK) that had been previously activated by passing through 4 ml methanol.

The cartridge was washed with 5 ml of 20 mM PBS and the 2,4-D was eluted from the cartridge with 6 ml methanol at a flow rate of 1 ml/min. The methanol was evaporated to dryness, the residue was resuspended in 2 ml methanol and filtered through a 0.22 μm filter prior to HPLC analysis.

To determine the rate of 2,4-D recovery, 5 g of plant material was spiked with 10 mg 2,4-D, refrigerated for 7 days and treated as described above.

HPLC was carried out on a C,8 polar end capped column (250×4.6 mm: Phenomenex, UK), with a mobile phase consisting of Methanol-Water-Acetic Acid (60:40:1), at a flow rate of 1 ml/min. Detection was by UV and 280 nm. A calibration curve was constructed using the integrator values obtained from the quantification of standard solutions. 20 μl standards or prepared samples were injected onto the column using a Rheodine injector system.

The rate of recovery of 2,4-D from spiked soil and plant tissue (after 7 days) was determined to be 100±10% and 98±5%, respectively.

Enumeration of P. putida VM1450 Within Plant Tissues

Plants were destructively sampled 4 days after the final addition of 2,4-D. Samples of leaf/stem, root and rhizosphere soil were taken from each plant. For surface sterilisation of the root and stem/leaf tissue, a sodium hypochloride solution (1% active chloride+200 μl/Tween 20) was used.

Leaves and stems were surface sterilised by submerging them in the sterilising solution for 5 min. They were then rinsed 3 times in sterile water. Roots were surface sterilised by placing them into the sterilising solution for 5 min, then rinsed 3 times in sterile water. This procedure was carried out twice on each root sample.

To check for sterility, surface sterilised tissues were pressed against a plate count agar (PCA) plate (Merck) and samples of the third rinsing were plated on PCA. 1 g of the surface sterilised tissues was homogenised using sterile pestle and mortars, serially diluted in 0.85% sterile saline and 100 μl samples were spread onto PCA containing 100 μg/ml kanamycin or 2,4-D plates (solid minimal media+1 mM 2,4-D). Rhizosphere samples were serially diluted and plated in the same manner.

Plates were incubated at 30° C. and examined for growth after 72 h. The number of colony forming units per gram (CFU/g) of fresh tissue was calculated. Sampling from each compartment and each treatment was carried out in triplicate.

In Planta Visualisation of P. putida VM1450 Using Epi-Fluorescence Microscopy

Hand cut sections of the root or surface sterilised stem and root tissues were stained with a 0.1% acridine orange solution for I min. The sections were then examined under blue light (395 nm) using a Nikon E400 epi-fluorescent microscope equipped with a 100 W mercury short arc photo-optic lamp and a FITC filer. Lucia® imaging software (version 4.6) was used to capture and process microscopic images.

The Natural Isolate P. putida VM1450 Helps Inoculated Plants to Maintain Growth and a Healthy Root System

The phyto-toxic effect of 2,4-D on the roots of non-inoculated plants were very clearly visible [FIG. 7A]. These plants developed enlarged root tips and thickening of the secondary roots. The root systems of non-inoculated plants showed severe callus formation and root thickening even at the lowest level of 2,4-D tested, whereas inoculated plants did not [FIG. 7B].

The presence of the inoculum had thus a profound protection effect on the root system of inoculated plants. Inoculated plants did display very minor toxicity symptoms but only at the highest level of 2,4-D tested (54 mg).

Results from the chlorophyll analysis showed that applications of 2,4-D lead to statistically significant reductions in chlorophyll a and b levels. The presence of P. putida VM1450 helped to maintain chlorophyll levels.

The ability of plants to maintain biomass is critical for phytoremediation. Maintaining a healthy root system and photosynthetic activity allows an increased water uptake through the roots and production of root exudates, which in turn results in a greater accumulation/uptake of pollutants both in the rhizosphere (where increased microbial activity can degrade the pollutant) and within the plant (where plant enzymes and edophytic populations can act on it).

These results indicate that the natural isolate P. putida VM1450 helps inoculated plants to maintain growth and a healthy root system in the presence of high levels of 2,4-D.

Degradation of 2,4-D from the Plant Tissues by P. putida VM1450

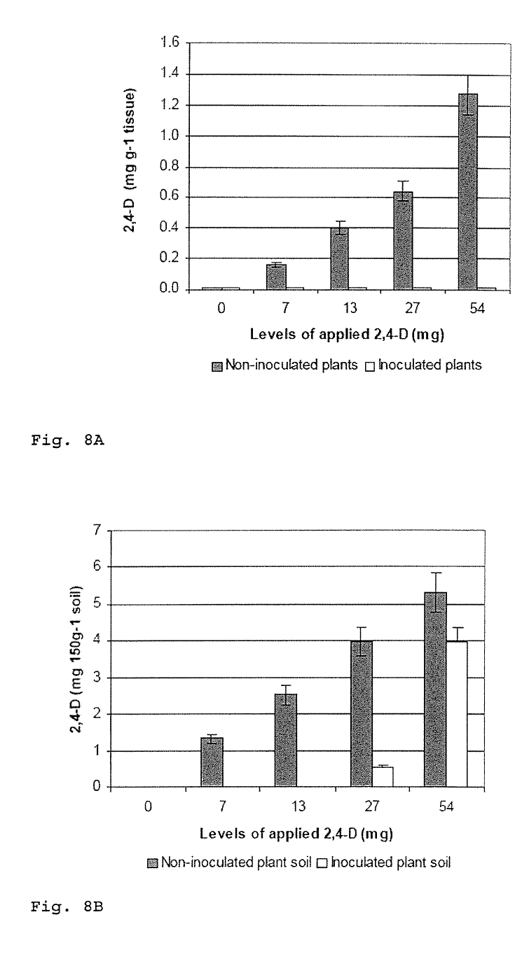

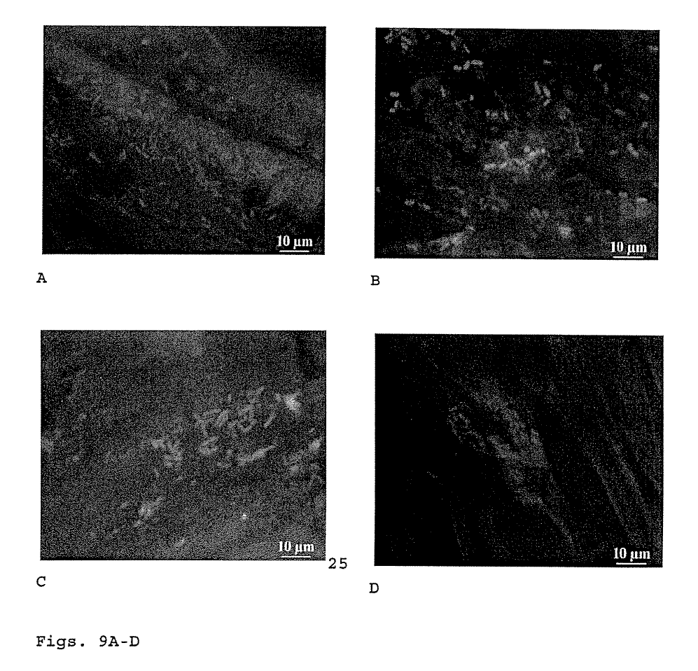

Accumulation of high levels of 2,4-D was observed within the stem/leaf tissue of 2,4-D treated non-inoculated plants [FIG. 8A]. This accumulation increased with corresponding increases in the level of applied 2,4-D and accounted for 24-35% of the total 2,4-D applied in the pots. Accumulation in the aerial tissue was expected as 2,4-D is a weak acid with a K_{ow}=2.083 allowing it to be readily transported to the leaves via the transpiration stream.

Pea plants inoculated with P. putida VM1450 did not show any accumulation of 2,4-D within their aerial stem/leaf tissue at any level of applied 2,4-D.

The fact that the roots displayed little or no symptoms of 2,4-D toxicity suggests that P. putida VM1450 populations in the rhizosphere and/or inside the root were partly responsible for the degradation of 2,4-D.

The pronounced reduction in chlorophyll levels of the leaf tissues indicates that considerable levels of 2,4-D were translocated to the aerial parts of the plants. The significant increase in the population of P. putida VM1450 and the inability to detect any 2,4-D within the stem-leaf tissue suggests that the VM1450 cell residing in these tissues degraded the translocated 2,4-D.

Enumeration of P. putida VM1450 cells in the various tissues of inoculated Pea plants showed that it efficiently colonized inoculated plants. Population sizes in the rhizosphere grew from an order of 105 with no selective pressure, to between 10⁶-10⁷ when 13-54 mg 2,4-D was added. The most noticeable change in population size was in the stem, where no detectable levels were obtained in inoculated plants that had received no 2,4-D.

Epi-fluorescence microscopy revealed that P. putida VM1450 resides in large bio-films on the rhizoplane and in micro-colonies within the root and stem tissues. Many micro-colonies and bio-films could be seen located in the rhizoplane [FIGS. 9A and 9B]. P. putida VM1450 cells could also be seen inside the root and stems, residing as discrete micro-colonies [FIGS. 9C and 9D] or inhabiting the intercellular spaces of these tissues.

The degradation of 2,4-D in the plant tissues by P. putida VM1450 is advantageous because it prevents the entry of 2,4-D into the food chain, thereby eliminating any toxic effects on herbivorous fauna residing in or near contaminated sites.

Removal of 2,4-D from the Soil

After plants were removed from the pots, soil samples were analyzed to determine the quantity of 2,4-D that remained in the soil. The results showed high levels of 2,4-D remaining in soil which held the non-inoculated plants [FIG. 8B]. These levels increased with the levels of added 2,4-D and accounted for 10-20% of the total 2,4-D applied to the pots. This quantity of 2,4-D along with the levels of recovered from the stem tissues accounted for 34-55% of the total 2,4-D applied to the pots. The remaining 45-66% is thought to have accumulated in the roots of non-inoculated plants, where 2,4-D caused severe damage to the root systems in control plants [FIG. 7].

This accumulation is not thought to have occurred in P. putida VM1450 inoculated plant roots as there were no visible signs of toxicity there. In soil, which had P. putida VM1450 inoculated plants, no remaining 2,4-D was detected in the pots that had received 7 or 13 mg 2,4-D.

At the two highest levels of applied 2,4-D (27 and 54 mg) there was still 2,4-D remaining in the soil of inoculated plants. However, the amount remaining accounted only for 1.8 and 7%, respectively, of the total 2,4-D level applied to these pots. These levels were significantly less than those remaining in the soil of non-inoculated plants.

In the present example, the plants were sampled 4 days after the final application (and highest dose) of 2,4-D. It is therefore likely that there was simply insufficient time for plant uptake of all the 2,4-D, the uptake of 2,4-D by the plant being the rate-limiting factor in 2,4-D removal from soil.

Conclusion

The endophytic strain protects pea (a broad leaf plant) from some of the toxic effects of high levels of the herbicide. This allows inoculated plants to increase their biomass and hence the plant uptake of 2,4-D from the soil. 2,4-D is quickly degraded inside the plant tissues by the root and stem colonizing endophytic bio-films and micro-colonies of P. putida VM1450. These data confirm the findings of Example 5 for toluene. These data further demonstrate that natural isolates equipped with the appropriate degradation pathway can be successfully used in the methods of the invention.

### Example 9

### Naphthalene and 2,4-D Phyto-Protection

The present example relates to phytoremediation of naphthalene using an endophytic micro-organism engineered to degrade this compound. The donor strain was Pseudomonas putida G7 carrying the NAH7 plasmid. The recipient endophyte strain P. putida POPHV9 was first marked with Km^{r}, Ni^{r} gfp cassette (Germaine et al 2003) to yield the strain VM1441. The donor G7pNAH7 was crossed with VM1441 by plate mating to yield the endegrader VM1441pNAH7.

The resulting strain P. putida VMM1441 containing the pNAH7 plasmid was then used as an inoculant of the model plant Pisum sativum var. Early Onward.

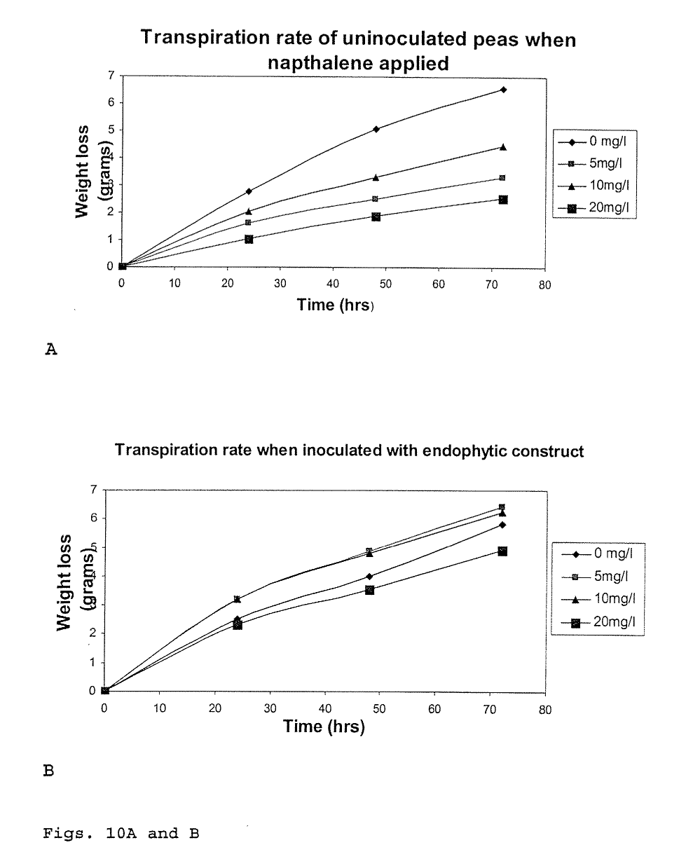

Increased protection of the plant from the toxic effects of naphthalene in growth medium was observed in inoculated plants compared to non-inoculated plants (FIGS. 10A and B). The general transpiration of the plant increased due to the effect that there is no toxicity of naphthalene.

FIG. 11 shows that there is a prominent phytoprotection against naphthalene when Pea plants are inoculated with P.putida VMM14441. The inoculated plants are clearly much healthier than the non-inoculated plants when grown in the presence of 10 mg/l naphthalene

The concept was also demonstrated for the herbicide 2,4-D, using an endophytic micro-organism capable of degrading this compound (see Example 8: P. putida VM1450).

Table 3 gives the percentage of inoculated and non-inoculated plants that survived in the presence of 1 mg/l 2,4-D (Pea) and 5 mg/l 2,4-D (Iris). As can be seen for Pea, 80% of the inoculated plants remained healthy whereas 80% of the control plants died. In the case of Iris, all inoculated plants remained healthy and survived the presence of 2,4-D well, whereas 75% of the control plants did not survive.

TABLE 3
Survival rate of Pea and Iris plants
in the presence and absence of 2,4-D.

| | |
|---|---|
| Non-inoculated | Inoculated |
| plants + 2,4-D | plants + 2,4-D |

| | Healthy | Dead | Healthy | Dead |
|---|---|---|---|---|
| % | plants | plants | plants | plants |
| Pisum sativum | 20 | 80 | 80 | 20 |
| Var. Early | | | | |
| Onward | | | | |
| Iris pseudocarus | 25 | 75 | 100 | 0 |

Phytoremediation of nitroaromatic compounds such as 2,4-DNT and TNT is possible by e.g. transferring the 2,4-DNT and TNT degradation pathways of Pseudomonas sp. ST53 (strain deposited in the BCCM-LMG culture collection in Ghent, Belgium) into related Pseudomonas sp. that show endophytic behavior.

Transfer of the 2,4-DNT and TNT degradation pathways can be obtained by natural gene transfer (see Examples 10 and 11). Many useful degradation pathways and degrader strains exist for these compounds. An example of a useful natural isolate is Methylobacterium populum sp., a methane utilizing bacterium isolated from poplar trees and able to degrade nitro-substituted explosives such as 2,4,6-Trinitrotoluene, Hexahydro-1,3,5-Trinitro-1,3,5-Triazine and Octahydro-1,3,5,7-Tetranitro-1,3,5-Tetrazocine (Van Aken, 2004a and b). The introduction of the degradation pathways results in improved in planta degradation of the nitroaromatic compounds.

### Example 10

### Horizontal Gene transfer of the Naphtalene Degradative Plasmid pNAH7 to Pea Endophytes

The present example shows that the naphthalene degradation pathway present on the plasmid pNAH7 (see Example 9) can be transferred to Pea endophytes via natural gene transfer. Naphthalene is the simplest example of a polycyclic aromatic hydrocarbon (PAH) that enters the environment from industrial uses e.g. a component of coal tar, from its use as a moth repellent, from the burning of wood or tobacco, and from accidental spills. Naphthalene at hazardous waste sites and landfills can dissolve in water. Naphthalene can become weakly attached to soil or pass through the soil into underground water and is volatile.

A total of 152 colonies were taken from minimal media and naphthalene selection plates. 72 colonies were isolated from plates where the plant was grown under selective pressure, and 80 colonies were isolated from plates where the plant was grown with no selection.

Colony PCR was carried out on all 152 colonies, 61 of which had the pNAH7 plasmid present. ERIC PCR was carried out on all 61 colonies. Their profiles were compared to those of the donor strain and 14 strains showed a different profile.

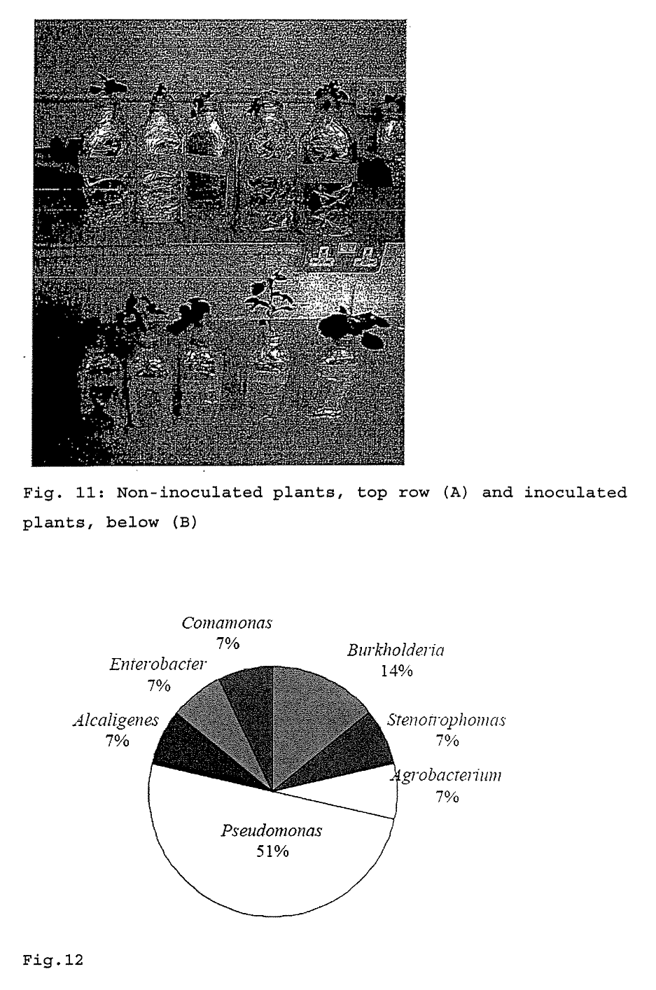

Biolog and 16sRNA gene analysis was carried out to identify the host endophyte (FIG. 12). The results indicate that the naphthalene degradation pathway present on the plasmid pNAH7 can be transferred to a wide variety of Pea endophytes via horizontal or natural gene transfer. The pNAH7 plasmid was found back in Burkholderia, Stenotrophomonas, Agrobacterium, Pseudomonas, Alcaligenes, Enterobacter and Comamonas endophytic bacteria isolated from Pea. The transfer frequency was estimated to be approx. 10⁻¹/recipient irrespective of the presence or absence of naphthalene.

Similar results were obtained with pTOM in Pea.

In conclusion, the results above show that horizontal gene transfer occurs at significant levels, even in the absence of selection as observed for amongst others pTOM and pNAH7.

### Example 11

### Horizontal Gene Transfer to Endogenous Endophtyic Bacteria from Poplar Improves Phytoremediation of Toluene

Materials and Methods

Strain Construction

The constitutively expressed toluene degradation pathway of B. cepacia BU61 (Shields and Reagin, 1992), a derivative of B. cepacia G4, was introduced by conjugation into B. cepacia BU0072 (Ni^{R}, Km^{R}) (Taghavi et al, 2001). Conjugation and selection of the transconjugants were performed as described previously (Barac et al, 2004). Transconjugants were selected on the basis of the acquired Ni^{r} Km^{r} Tol⁺phenotype on 284 minimal medium (Mergeay, 1985) supplemented with 1 mM NiCl₂ and 100 μg/ml kanamycin while the plates were incubated under a toluene atmosphere as a carbon source. The presence of the nre Ni resistance operon and the pTOM plasmid in the transconjugants was confirmed by PCR using nre- and pTOM-specific primers, respectively. The primers specific for tomA4 (accession number AF319657), forward primer 5′-GTTGCCCTCAAACCCTACAA-3′ (position 3323) and reverse primer 5′-AGGGGCTGAATGTTGAGTTG-3′ (position 3780), amplified a 458-bp fragment; the primers specific for nreB (accession number L31491), forward primer 5′-GGATTACCGAGCCAGTTTCA-3′ (position 2421) and reverse primer 5′-GGTGTCTGCGTCATCGAATA-3′ (position 3445), amplified a 1,025-bp fragment. In addition to the presence of nre and pTOM, BOX-PCR (Vinuesa et al, 1998) was used to confirm the genetic background of the transconjugants. A representative transconjugant, B. cepacia strain VM1468, which grew under the appropriate selective conditions (1 mM NiCl₂, 100 μg/ml kanamycin) with toluene as the sole carbon source, was selected for further study.

Microbial Inoculation of Poplar Cuttings

Cuttings were taken from P. trichocarpa×deltoides cv. Hoogvorst. The cuttings were 40 cm long and had a diameter of approximately 1 cm. The cuttings were surface disinfected with 75% ethanol and placed with their bottom ends in tap water for 4 weeks to allow establishment of roots, after which root inoculation was performed.

B. cepacia strains VM1468 and BU61, which were used as inocula, were grown for 24 h in 284 gluconate medium at 30° C. on a rotary shaker. After the cultures reached an optical density at 660 nm of about 1, which corresponded to 10⁹ CFU/ml, cells were harvested by centrifugation and subsequently washed twice with 10 mM MgSO₄. The inoculum was prepared by suspending the cells to a final concentration of 10⁸ CFU/ml in a solution comprised of 1 liter of half-strength sterile Hoagland's nutrient solution (Barac et al, 2004) to which 200 ml of 284 gluconate medium was added. Cuttings were placed with their roots in the inoculum for 72 h, while control plants were placed in the same solution without bacteria for 3 days. Subsequently, the cuttings were planted in 4-liter pots which were filled with nonsterile sandy soil and saturated with half-strength Hoagland's solution. Before planting the weights, numbers of roots and leaves, and root lengths of the cuttings were determined. The plants were allowed to stabilize for 2 weeks under greenhouse conditions, during which they were watered every other day with half-strength Hoagland's solution or distilled water; after this they were challenged with toluene.

Effect of Toluene on the Growth of Poplar Cutting

Starting 2 weeks after they were transferred into sandy soil, the poplar cuttings inoculated with B. cepacia VM1468 or BU61 or the noninoculated control plants were watered for 10 weeks with half-strength Hoagland's solution containing 500 mg/liter toluene, which resulted in gradual exposure of the plants to toluene. At the beginning of the experiment, the weight of a cutting together with the mass of its pot plus the sandy soil irrigated with Hoagland's solution to field capacity was adjusted to 4,800 g. Every other day, pots with cuttings were weighed and irrigated until they were their original weight with the half-strength Hoagland's solution to which toluene was added at a concentration of 500 mg/l. Control experiments without toluene were also set up.

In order to prevent growth of algae, the pots were covered with dark gray plastic foil. For each treatment 10 replicas were established. After 10 weeks the cuttings were harvested, and the following growth parameters were determined: total biomass, number of roots, root weight and length, number of young twigs, number of leaves, leaf surface area, and total leaf weight.

Recovery of Inoculated Bacteria.

At the end of the toluene exposure experiment plants were harvested, and the microbial colonization of the plants was examined. In order to examine the rate of successful inoculation, rhizosphere, root, stem, twig, and leaf samples were taken from inoculated and control plants that had been watered with Hoagland's solution containing either 0 or 500 mg/l toluene. Roots and leaves were vigorously washed in distilled water for 5 min and surface sterilized for 10 min in a solution containing 2% (wt/vol) active chloride added as an NaOCl solution (Fluka) and supplemented with I droplet of Tween 80 (Merck) per 100 ml of solution; stems and twigs were vigorously washed in distilled water for 5 min and sterilized for 5 min in a solution containing 1% active chloride supplemented with 1 droplet of Tween 80 (Merck) per 100 ml of solution. After surface sterilization leaves, roots, stems, and twigs were rinsed three times for 1 min in sterile water and dried on sterile filter paper. The water from the third rinse was plated on 869 medium (Mergeay, 1985) as a control for sterility. After surface sterilization plant parts were macerated in 10 mM MgSO₄ using a Polytron PT1200 mixer (Kinematica A6). Serial dilutions were made, and 100-μl samples were plated on 284 medium to which either gluconate or toluene was added as a carbon source in order to test for the presence of the inoculated strains, as well as other cultivable bacteria. The rhizosphere samples were serially diluted in 10 mM MgSO₄ and plated on the same media. After 7 days of incubation at 30° C. the number of CFU was determined and expressed per gram of plant tissue or rhizosphere soil.

Morphologically different bacteria that grew on toluene as a sole carbon source were purified three times on 284 gluconate medium or on 10-fold-diluted 869 medium, after which growth on toluene was confirmed, as was the ability to grow autotrophically with CO₂ as the carbon source. Tol⁺ strains were genetically fingerprinted using BOX-PCR (Vinuesa et al, 1998). The bacteria that exhibited different BOX-PCR patterns and that were able to grow on toluene as a sole carbon source were tested for the presence of the pTOM plasmid by PCR with tomA4-specific primers and were further identified by sequencing their 16S rRNA genes using the standard 26F-1392R primer set (Amann et al, 1995).

Toluene Evapotranspiration.

Poplar cuttings (P. trichocarpa×deltoides cv. Hoogvorst), inoculated as described above, were used to evaluate the phytotoxicity and in planta degradation of toluene. Non-inoculated plants were examined as controls. Poplar is a preferred plant for phytoremediation of contaminated aqueous media.

After 7 weeks of growth under greenhouse conditions, the cuttings were carefully taken out of the jars, and their roots were vigorously rinsed in sterile water to remove bacteria from the surface. Subsequently, plants were grown hydroponically in a two-compartment glass cuvette system (height, 60 cm; diameter, 25 cm) (Barac et al, 2004). Each cuvette compartment was inserted into a flowthrough system using a synthetic air source (Air Liquide) with an inflow of 3 liters per h on one side and with two linked Chromosorb 106 traps (capped sample tubes [Perkin-Elmer] and Chromosorb 60/80 [Alltech]) on the outflow. A column with CaCl₂ was placed between the plant cuvettes and the Chromosorb traps in order to prevent water condensation in the traps. The lower compartment was filled with 2.5 liters of half-strength sterile Hoagland's solution to which 100 mg/l toluene was added at the beginning of the experiment. The Chromosorb traps were changed at regular intervals, and they were analyzed by gas chromatography-mass spectrometry (GC-MS) with an ATD400 automatic thermal desorption system, an Auto System XLL gas chromatograph, and a Turbo mass spectrometer (Perkin-Elmer). To avoid gas exchange between the upper and lower compartments, the compartments were separated by a glass plate with an insert through which the stem of the plant was introduced. Each cuvette contained one plant, and the space around the stem was made gas tight with a Polyfilla exterior mixture (Polyfilla), so that the shoots in the upper compartment and the roots in the lower compartment were completely separated; this allowed no gas exchange between the shoots and the roots except through the stem. The upper compartment, the glass plate, and the lower compartment were sealed with Apiezon (Apiezon Products M&I Materials Ltd.).

Cuvettes with plants were placed in a growth chamber with a constant temperature of 22° C. and a cycle consisting of 14 h of light (photosynthetically active radiation; 165 μmol m⁻² s⁻¹) and 10 h of darkness. The whole experiment was performed for 96 h, and the toluene concentrations in the traps were determined by GC-MS. The amount of evapotranspired toluene was calculated per unit of leaf surface area. All experiments were performed in triplicate to allow statistical analysis of the data using analysis of variance.

Toluene Phytotoxicity: Effect on the Growth of Poplar Cuttings

Poplar cuttings were inoculated with B. cepacia strain VM1468 or BU61 and grown in conditions under which they were watered with half-strength Hoagland's solution to which 500 mg/l toluene was added. This resulted in step-wise wash-in of toluene. This high toluene concentration was chosen so that there would be clear effects of toluene toxicity in a relatively short time.

VM1468 and BU61 both possess the pTOM-Bu61 plasmid that constitutively expresses toluene degradation. Yet VM1468 is an endophyte, whereas BU61 is a soil isolate that is not able to enter and colonize the plant [poplar].

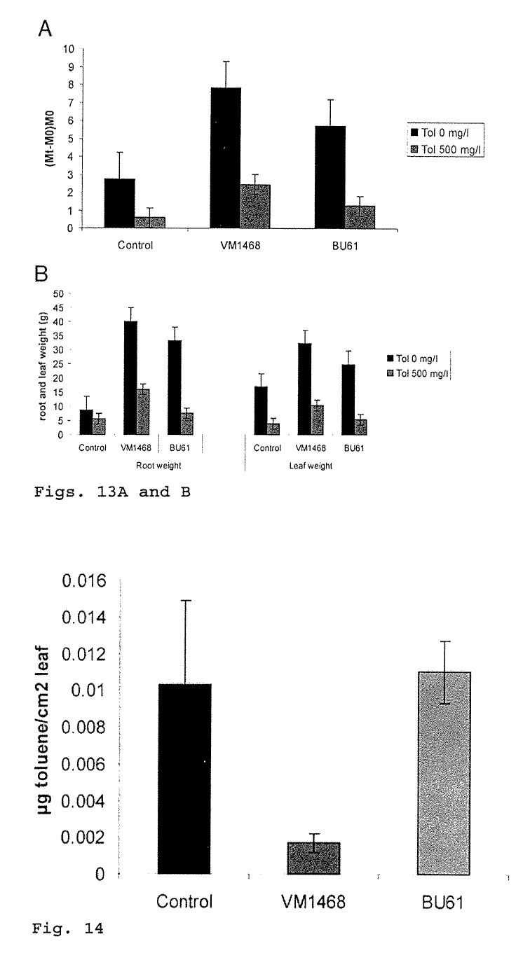

The effect on plant's fresh weight, root and leaf weight was evaluated. The results are presented in FIGS. 13A and 13B.

Both strains had a growth promoting effect: plants inoculated with either bacterial strain produced more biomass than the non-inoculated plants, even in the absence of toluene. Yet, importantly, plants reinoculated with VM1468 produced significantly more biomass than the non-inoculated plants and the plants inoculated with B. cepacia BU61. The biomass produced by plants inoculated with B. cepacia BU61 was 2 times the biomass by the control plants but only one-half the biomass produced by plants inoculated with VM1468.

All plants exposed to toluene produced significantly less root biomass than plants grown in the absence of toluene. After toluene exposure, the root biomass for the non-inoculated plants and the plants inoculated with B. cepacia BU61 did not differ significantly. Plants inoculated with B. cepacia VM1468 on the other hand produced about twice as much root biomass.

The effects of toluene exposure and microbial inoculation were also significant when leaf parameters were analyzed. After toluene treatment, plants inoculated with B. cepacia VM1468 formed 30 to 40% more leaves than plants inoculated with B. cepacia BU61, and about 60% more leaves than non-inoculated control plants formed (results not shown).

Toluene Evapotranspiration

Seven weeks after inoculation with B. cepacia VM1468 or BU61, poplar plants were challenged by adding toluene at a sub-phytotoxic concentration (100 mg/l). After 96 h the total amount of toluene that evapotranspirated through the aerial parts of the plants (upper compartment) was measured using GC-MS. The results for toluene evapotranspiration, expressed on the basis of leaf surface area, are shown in FIG. 14.

Compared to non-inoculated plants or plants inoculated with B. cepacia BU61, poplar cuttings inoculated with B. cepacia VM1468 released about 5 times less toluene through the leaves. It is important to notice that plants inoculated with B. cepacia VM1468 or BU61 had similar biomasses (63 g±(g), making it very unlikely that the observed differences in toluene evapotranspiration resulted from binding of toluene to the plant biomass.

This illustrates that inoculation with endophytic bacteria possessing the right degradation pathway not only protects plants against toluene phytotoxicity but also reduces the evapotranspiration of the pollutant into the air. These data confirm the data of Example 5, in which another strain was used. It should be noted that poplar is not the natural host of the inoculating strain used here.

Recovery of Inoculated Bacteria

Since the natural host plant of B. cepacia is yellow lupine, the colonization of poplar by this bacterium was examined. To do this, poplar cuttings were inoculated with B. cepacia VM1468 and grown for 12 weeks under greenhouse conditions in the presence or absence of 500 mg/l toluene, after which the rhizosphere, roots, stems, twigs and leaves were sampled to study the presence of the inoculum. Plants that did not receive an inoculum and plants inoculated with B. cepacia BU61 were used as controls. Seven days after samples were plated on different media, the total number of bacteria, their morphology, and their ability to grow on toluene as a sole carbon source were determined (Table 4). The number of CFU was calculated per gram (fresh weight) of plant material or rhizosphere soil.

Cultivable endophytic bacteria were found in all parts of inoculated and non-inoculated poplar plants. For non-inoculated poplar, only 1 type of rhizosphere bacteria was found to be able to grow on toluene as a sole carbon source, while no cultivable endophytic bacteria able to grow on toluene as a sole carbon source were observed. For plants inoculated with B. cepacia VM1468 or BU61, both rhizosphere and enodphytic bacteria able to grow on toluene as sole carbon source were isolated.

TABLE 4
Recovery of cultivable bacteria from P. trichocarpa × deltoides
cv. Hoogvorst cuttings which were inoculated with
B. cepacia VM1468 or BU61. The microbial populations
from noninoculated plants were analyzed as controls.
Bacteria were isolated on 284 medium plates to which
toluene or gluconate was added as a sole carbon source.
The numbers of bacteria isolated on the different
growth media are expressed per gram (fresh weight)
of rhizosphere soil or plant material.

| | | Toluene | | |
|---|---|---|---|---|
| | | concn | No. of cells/g (fresh wt) on: | |
| Inoculum | Sample | (mg/liter) | 284 + gluconate | 284 + toluene |
| None | Rhizosphere | 0 | 2.5 × 107 | 2.2 × 108 |
| None | Root | 0 | 9.6 × 109 | 0 |
| None | Stem | 0 | 1.1 × 109 | 0 |
| None | Twig | 0 | 4.5 × 108 | 0 |
| None | Leaf | 0 | 5.9 × 107 | 0 |
| None | Rhizosphere | 500 | 2.1 × 107 | 8.9 × 107 |
| None | Root | 500 | 3.3 × 108 | 0 |
| None | Stem | 500 | 3.1 × 108 | 0 |
| None | Twig | 500 | 3.2 × 108 | 0 |
| None | Leaf | 500 | 2.9 × 106 | 0 |
| VM1468 | Rhizosphere | 0 | 2.1 × 107 | 1.2 × 107 |
| VM1468 | Root | 0 | 4.3 × 108 | 3.8 × 108 |
| VM1468 | Stem | 0 | 5.2 × 108 | 9.2 × 106 |
| VM1468 | Twig | 0 | 3.2 × 104 | 3.7 × 104 |
| VM1468 | Leaf | 0 | 6.8 × 102 | 0 |
| VM1468 | Rhizosphere | 500 | 1.7 × 107 | 1.2 × 107 |
| VM1468 | Root | 500 | 5.6 × 108 | 5.6 × 107 |
| VM1468 | Stem | 500 | 4.6 × 107 | 2.2 × 108 |
| VM1468 | Twig | 500 | 3.6 × 108 | 2.1 × 106 |
| VM1468 | Leaf | 500 | 1.2 × 108 | 0 |
| BU61 | Rhizosphere | 0 | 5.4 × 106 | 3.2 × 107 |
| BU61 | Root | 0 | 4.8 × 108 | 2.5 × 108 |
| BU61 | Stem | 0 | 6.6 × 108 | 2.1 × 108 |
| BU61 | Twig | 0 | 0 | 0 |
| BU61 | Leaf | 0 | 6.4 × 107 | 0 |
| BU61 | Rhizosphere | 500 | 7.0 × 106 | 7.5 × 106 |
| BU61 | Root | 500 | 2.7 × 108 | 1.6 × 108 |
| BU61 | Stem | 500 | 3.2 × 108 | 2.9 × 108 |
| BU61 | Twig | 500 | 3.7 × 108 | 4.1 × 108 |
| BU61 | Leaf | 500 | 2.7 × 107 | 0 |

This result indicates that the presence of Tol⁺ bacteria depends on the presence of the inoculum, despite the fact that Tol⁺ bacteria could be isolated from the rhizosphere of noninoculated control plants. In addition, toluene selection pressure had no effect on the numbers of Tol+bacteria in the endophyte community.

Interestingly, based on their morphology, different endophytic bacteria were found to grow on toluene as a carbon source. Since no toluene-degrading endophytes were found in the control plants, this strongly suggests that horizontal gene transfer of the tom operon occurred from B. cepacia VM1468 and BU61 to the natural endophytic communities of poplar.

Horizontal Gene Transfer of the Degradation Genes to the Natural Endophytic Communities

To test the hypothesis of horizontal or natural gene transfer to the natural endophytic communities, morphologically different bacteria growing on media with toluene as a sole carbon source were characterized further. Their DNAs were extracted, the strains were fingerprinted using BOX-PCR, and PCR with tomA4-specific primers was used to confirm the presence of the tom operon. In addition, the 16S rRNA genes of strains that had different BOX-PCR fingerprints were PCR amplified, cloned and sequenced. As a control to demonstrate horizontal gene transfer of the tom operon, one also tried to identify the corresponding recipients among the cultivable bacteria from the non-inoculated control plants.

The results for representative strains are shown in Table 5. Tom-mediated growth on toluene was observed for bacteria belonging to different branches of the Proteobacteria. The most common species found were Enterobacter sp., Pseudomonas putida, and Stenotrophomonas maltophilia.

All bacteria tested showed BOX-PCR fingerprints that differed from those of the B. cepacia VM1468 and BU61 donor strains; this included B. cepacia T675, which was isolated from poplar inoculated with strain VM1468.

This finding indicates that the inoculum strains were only a minor fraction of the endogenous endophytic community of poplar, if they were able to establish themselves at all. Strains with identical BOX-PCR patterns could be found among rhizosphere and endophytic isolates that had acquired the tom toluene degradation pathway by horizontal gene transfer. This horizontal gene transfer of the plasmid pTOM-Bu61 happened both in the presence and in the absence of toluene.

Despite the fact that both B. cepacia BU61 and VM1468 were able to transfer the pTOM-Bu61-encoded tom operon to the endogenous microbial populations associated with poplar, major differences were observed. Plants inoculated with VM1468 suffered much less from toluene toxicity and released less toluene into the environment, indicating that their microbial communities were better adapted to degrade toluene than plants inoculated with soil isolate BU61. This observation might be explained by the endophytic character of strain VM1468. Strain BU61 is unable to enter poplar as an endophyte. Therefore, BU61 can act only as a donor strain to transfer the tom operon to bacteria present in the poplar rhizosphere.

TABLE 5
Representative rhizosphere and endophytic strains found in association
with poplar plants that were inoculated with B. cepacia BU61 or VM1468.

| | | | Toluene | tomA4 | |
|---|---|---|---|---|---|
| Strain^{a} | Origin | Species | phenotype | PCR | BOX-PCR pattern |
| BU61 | Control | B. cepacia | Tol⁺ | + | Unique |
| VM1468 | Control | B. cepacia | Tol⁺ | + | Unique |
| W604 | Root, VM1468 | Enterobacter sp. | Tol⁺ | + | Similar to S662, S671, |
| | inoculum | | | | R558-1 |
| W607 | Root, VM1468 | Stenotrophomonas | Tol⁺ | + | Similar to S649, W635, |
| | inoculum | maltophilia | | | R551-3 |
| W619 | Root, VM1468 | Pseudomonas | Tol⁺ | + | Similar to W630, |
| | inoculum | putida | | | W645, S672, R599 |
| W630 | Root, BU61 | Pseudomonas | Tol⁺ | + | Similar to W619, |
| | inoculum | putida | | | W645, S672, R599 |
| W633 | Root, BU61 | Ochrobactrum sp. | Tol⁺ | + | Unique |
| | inoculum | | | | |
| W635 | Root, BU61 | Stenotrophomonas | Tol⁺ | + | Similar to W607, S649, |
| | inoculum | maltophilia | | | R551-3 |
| S645 | Stem, VM1468 | Pseudomonas | Tol⁺ | + | Similar to W619, |
| | inoculum | putida | | | W630, S672, R599 |
| S649 | Stem, VM1468 | Stenotrophomonas | Tol⁺ | + | Similar to W607, |
| | inoculum | maltophilia | | | R551-3 |
| S656 | Stem, VM1468 | Stenotrophomonas | Tol⁺ | + | Similar to R572, R591 |
| | inoculum | maltophilia | | | |
| S662 | Stem, VM1468 | Enterobacter sp. | Tol⁺ | + | Similar to W604, S671, |
| | inoculum | | | | R558-1 |
| S671 | Stem, BU61 | Enterobacter sp. | Tol⁺ | + | Similar to W604, S662, |
| | inoculum | | | | R558-1 |
| S672 | Stem, BU61 | Pseudomonas | Tol⁺ | + | Similar to W619, |
| | inoculum | putida | | | W630, S645, R599 |
| T675 | Twig, VM1468 | B. cepacia | Tol⁺ | + | Unique |
| | inoculum | | | | |
| R571 | Rhizosphere, VM1468 | Pseudomonas | Tol⁺ | + | Unique |
| | inoculum | putida | | | |
| R572 | Rhizosphere, VM1468 | Stenotrophomonas | Tol⁺ | + | Similar to S656, R591 |
| | inoculum | maltophilia | | | |
| R591 | Rhizosphere, | Stenotrophomonas | Tol⁺ | + | Similar to R572, S656 |
| | BU61 inoculum | maltophilia | | | |
| R599 | Rhizosphere, | Pseudomonas | Tol⁺ | + | Similar to W619, |
| | BU61 inoculum | putida | | | W630, S645, S672 |
| R551-2 | Rhizosphere, no | Pseudomonas | Tol⁺ | − | Unique |
| | inoculum | putida | | | |
| R551-3 | Rhizosphere, no | Stenotrophomonas | Tol⁻ | − | Similar to W607, |
| | inoculum | maltophilia | | | W635, S649 |
| R557-2 | Rhizosphere, no | Pseudomonas sp. | Tol⁻ | − | Unique |
| | inoculum | | | | |
| R558-1 | Rhizosphere, no | Enterobacter sp. | Tol⁻ | − | Similar to W604, |
| | inoculum | | | | S662, S671 |
| R558-2 | Rhizosphere, no | Pseudomonas | Tol⁻ | − | Unique |
| | inoculum | putida | | | |

Newman (Newman and Reynolds, 2005) postulated that several obstacles had to be overcome before the technology of using (engineered) endophytic bacteria to improve phytoremediation of volatile organic contaminants could move towards application. The major concern one saw in the persistence and stability of the engineered organisms and their degradation capabilities in field-grown plants, as phytoremediation projects can conceivably last decades.

The above results demonstrate that it does not matter that the initial bacteria of the inoculum become an integrated part of the endogenous endophytic community or not.

Horizontal gene transfer (HGT) has been shown to play an important role in allowing a microbial community to rapidly adapt to a new environmental stress. HGT now appears to play an important role in adapting the endogenous endophytic community (van der Lelie et al, 2005), rather than integrating a new bacterium into a stable community. The degradation pathway is as such transferred among the members of the community, rapidly and efficiently, despite the fact that that the inoculating strain is unable to establish itself eventually.

The above data also show that the desired metabolic properties could be transferred to a wide range of endogenous endophytic bacteria. The transfer of the desired properties into the endophytic community of a plant depends mainly on the efficiency of endophytic colonization of the transconjugants.

The observation that there is horizontal gene transfer opens the possibility of direct adaptation of a plant's endogenous endophytic population without the need of first selecting the appropriate endophytic micro-organism from the plant species of interest. In order to be successful, the genetic information encoding the desired metabolic properties should then be present on a broad-host range plasmid that can be efficiently transferred within the endophytic community, and that preferably has a broad expression range.

There are a few advantages linked to this approach: there is no need to isolate plant-specific endophytic bacteria; there is no need for genetic manipulation of isolated plant-specific endophytes; and there is no need to establish the endophytic inoculum in the plant's endogenous endophtyic community as the genetic information will be transferred to many members of the endogenous endophytic population.

As long as selection pressure is present, there will be a selective advantage for the endophytic population possessing the appropriate degradation characteristics. Preferably, during the construction of the remediation site appropriate measures are taken to maintain the selection pressure on trees (plants) inoculated with an endophytic community possessing the desired metabolic capacities. For instance, below-ground irrigation of plants with contaminated groundwater may be used to maintain the appropriate selection pressure until plant (tree) roots arrive at the level of the contaminated groundwater.

Maintaining the selection pressure is no guarantee that the endopythic inoculum will become an integrated part of the endophytic community, yet as shown above, horizontal gene transfer seems to occur at high frequencies, even in the absence of selection pressure. In that case, it does not matter that the endophytes of the inoculum are not able to establish themselves.

The endophytic inoculum or the broad-spectrum plasmid used can be seen as a starter culture.

General Conclusion

Endophytic organisms equipped with degradation and/or treatment genes are able to protect a plant from phytotoxic effects and are able to improve its phytoremediation capacities.

If the endophyte is able to degrade an organic pollutant inside the plant, the plant automatically benefits therefrom. Aim of the methods of the invention is to complement the metabolism of the host.

Both natural isolates and strains engineered to contain the appropriate genes may be used in a method of the invention.

Endophytes own to the plant are able to establish themselves within the endogenous community. However, if plasmids or mobile elements are introduced that are stable and compatible with those of the receptor strain and the endogenous endophytes, horizontal gene transfer is possible.

If horizontal gene transfer (HGT) occurs, it does not matter that the inoculating strain is not able to establish itself as the desired properties are passed on to the endogenous community of the plant.

The principle of HGT advantageously can be exploited to directly adapt the metabolic capabilities of a plant's endogenous endophytic community. Preferably measures are taken to maintain a selection pressure on plants (trees) inoculated with an endophytic community. Selection pressure means a selective advantage for the endophytic population.

The system of the present invention is generally applicable and works not only with test plants but also with plants like willow and poplar, plants commonly used in phytoremediation.

A deposit of the strain Burkholderia cepacia L.S.2.4::ncc-nre has been made according to the Budapest Treaty under the deposit number LMG P-20359 at the BCCM/LMG, Laboratorium voor Microbiologie—Bacteriënverzameling, Universiteit Gent, K. L. Ledeganckstraat 35, B-9000 Gent, Belgium on May 3, 2001.

### REFERENCES, INCORPORATED HEREIN BY REFERENCE IN THEIR ENTIRETY

Arazi, T. et al. The Plant Journal 20: 171-182 (1999).
Brewer, E. P. et al. Theo. Appl. Genet. 99: 761-771 (1999).
Burd, G. I. Appl. and Environ. Microbiol. 64: 3663-3668 (1998).
Cunningham, S. D. et al. In Vitro Cell. Dev. Biol. 29: 207-212 (1993).
Cunningham, S. D. et al. Plant Physiol. 110: 715-719 (1996).
De Souza, M. L. et al. Appl. Environ. Microbiol. 64: 2323-2326 (1998).
Diels, L. et al. J. Ind. Microbiol. 14:142-153 (1995).
Don, R. H. and Pemberton, J. M. J. Bacteriol.145: 681-686 (1981).
Don, R. H. and Pemberton, J. M. J. Bacteriol.466-468 (1985).
Di Fiore, S. and Del Gallo, M. Azospirillum VI and related microorganisms Springer Verlag, Berlin, Heidelberg (1995).
Hebbar, K. P. et al. Soil Biol. Biochem. 24: 989-997 (1992a).
Hebbar, K. P. et al. Soil Biol. Biochem. 24: 999-1007 (1992b).
Kloepper, J. W. et al. Can. J. Microbiology 38, 1219-1232 (1992).
Kloepper, J. W. et al. Phytopathol. 82: 726-727 (1992).
Mc Crady J. et al. J. Experimental Botany 38: 1875-1890 (1987).
Mergeay, M. et al. J. Bacteriol 162: 328-334 (1985).
Mergeay, M. 1997. Microbial resources for bioremediation of sites polluted by heavy metals. J. Wild et al.(eds) Perspectives in Bioremediation, 65-73. Kluwer Academic Publishers.
Pfleeger, T. et al. A short-term bioassay for whole plant toxicity. In: Plants for Toxicity Assessment: Second Volume, ASTM STP 1115. J. W. Gorsuch, R. W. Lower, W. Wang and M. A. Lewis (Eds.), American Society for Testing and Materials, Philadelphia, pp. 355-364 (1991).
Salt, D. E. et al. Int. J. Phytorem. 1: 67-79 (1999).
Shields, M. S. and Reagin, M. J. Appl. Environ. Microbiol. 58: 3977-3983 (1992).
Shields M S Appl. Environ. Microbiol. 61: 1352-1356 (1995).
Szpirer, C. et al. Microbiol. 145: 3321-3329 (1999).
Taghavi, S. Res. Microbiol. 148: 536-551 (1997).
Taghavi, S. et al. Appl and Environ Microbiol 67: 1015-1019 (2001).
Thompson P. L. et al. Environ. Toxicol. Chem. 17: 902-906 (1998).
Trapp, S. Environmental Toxicology and Chemistry 13 (3): 413-422 (1994).
Trapp, S. Pest Management Science (formerly Pesticide Science) 56: 767-778 (2000).
Trapp S. Environ. Sci. Technol., in print (2001).
Lodewijckx, C. et al. Int. J. Phytorem. 3: 173-187 (2001).
Germaine, K. E. et al. FEMS Microbiol Ecology 48:109-118 (2004)
Huang, X. D. et al. Evironm Pollut. 130:453-463 (2004).
Barac, T. S. et al. Nat Biotechnol. 22:583-588 (2004).
Vinuesa, P., et al. Appl Envrion Microbiol. 64:2096-2104 (1998)
Amann, R. I. et al. Microbiol Rev. 59:143-169 (1995).
Newman, L. A. & Reynolds, C. M. Trends Biotechnol. 23:6-8; discussion 8-9 (2005).
van Der Lelie, D. et al. Trends Biotechnol. 23:6-8 (2005).
Van Aken, B. et al. Int J Syst Evol Microbiol 54:1191-1196 (2004a).
Van Aken, B. et al. Appl Environ Microbiol 70:508-517 (2004b).
Germaine, K. E. et al. (2003).

1. A method for the phytoremediation treatment of a medium contaminated with an organic pollutant, the method comprising
selecting an endophytic microorganism containing degradation genes for the organic pollutant;
cultivating upon the contaminated medium a plant associated with an endophytic microorganism able to improve the phytoremediation of the organic pollutant by the plant.

2. The method according to the claim 1, wherein the endophytic microorganism is an endophytic bacteria.

3. The method of claim 2, wherein the endophytic bacterium is a naturally isolated endophytic bacterium.

4. The method of claim 1, wherein the endophytic microorganism is a genetically modified endophytic microorganism.

5. The method of claim 2, wherein the endophytic bacteria is a genetically modified endophytic bacteria.

6. The method of claim 1, wherein the endophytic micro-organism contains degradation genes for toxic metabolic degradation products of the pollutant.

7. The method according to the claim 1, wherein the contaminated medium is a contaminated soil or a contaminated aqueous medium.

8. The method according to claim 1, wherein the organic pollutant is water soluble.

9. The method according to claim 1, wherein the organic pollutant is volatile.

10. The method according to claim 9, wherein the volatile organic pollutant is MTBE, BTEX, TCE or PER.

11. The method according to claim 1, wherein the organic pollutant is a herbicide.

12. The method according to the claim 11, wherein the agrochemical is 2,4-Dichlorophenoxyacetic acid.

13. The method according to claim 1, wherein the organic pollutant is a nitroaromatic compound.

14. The method according to claim 13, wherein the organic pollutant is selected from the group consisting of 2,4-DNT, TNT, and amido-dinitrotoluene.

15. The method according to claim 1, wherein the organic pollutant is selected from the group consisting of phenol, chlorophenol, toluene, benzene, and naphthalene.

16. The method according to claim 1, wherein the organic pollutant is selected from the group consisting of chlorinated ethenes, organotin compounds, PCBs, PBBs, brominated flame retardants, and fluorinated alkylsulfonates.

17. The method according to claim 1, wherein the organic pollutant is perfluoro-octanyl sulfonate (PFOS).

18. The method according to claim 1, wherein the endophytic microorganism is present in the vascular system and/or the roots of the plant.

19. The method according to claim 1 additionally comprising transferring the degradation genes of the endophytic micro-organism are transferred to least one endophytic micro-organism of the plant's endogenous endophytic community.

20. The method according to claim 1 additionally comprising applying selection pressure as selective advantage for plants associated with the endophytic micro-organism.

21. A plant comprising in its vascular and/or root system a genetically modified endophytic microorganism able to express proteins allowing the degradation of the organic pollutant in planta.

22. A method for adapting the metabolic capabilities of a plant's endogenous endophytic population for phytoremediation of an organic pollutant, the method comprising
equipping a donor strain with broad-host-range plasmid having degradation genes for an organic pollutant; and
contacting the plant with the donor strain containing the plasmid for transfer of the degradation genes within the endogenous endophytic community of the plant; and
applying selection pressure to the plant in favor of transferring the degradation genes;
wherein the metabolic capabilities of the a plant's endogenous endophytic population is adapted for phytoremediation of the organic pollutant.

23. The method of claim 22, wherein this transfer occurs via horizontal gene transfer.

24. The method of claim 22, wherein the plasmid has a broad expression range.

25. A plant comprising in its endogenous endophytic community at least one endophytic species that has received, via horizontal gene transfer, degradation genes for an organic pollutant from an endophytic micro-organism with which the plant was associated.


SEQUENCE LISTING (6 sequences)

SEQ ID NO: 1 - DNA, 53 bp, Artificial
  BU0072 x G4 transconjugants confirmation sense primer
ggagagcgccgtgacccaggcgaagaaggcgctggtgcatgaccatatcgacc

SEQ ID NO: 2 - DNA, 58 bp, Artificial
  BU0072 x G4 transconjugants confirmation antisense primer
cacgatcaccatggcgctggctgctgccgcggcaagattcagcgccagcagtcccttc

SEQ ID NO: 3 - DNA, 20 bp, Artificial
  tomA4 forward primer
gttgccctcaaaccctacaa

SEQ ID NO: 4 - DNA, 20 bp, Artificial
  tomA4 reverse primer
aggggctgaatgttgagttg

SEQ ID NO: 5 - DNA, 20 bp, Artificial
  nreB forward primer
ggattaccgagccagtttca

SEQ ID NO: 6 - DNA, 20 bp, Artificial
  nreB reverse primer
ggtgtctgcgtcatcgaata